(19) United States
(12) Patent Application Publication
(10) Pub. No.: US 2025/0002854 A1
(43) Pub. Date: Jan. 2, 2025

(54) METHODS OF GENERATING POPULATIONS OF TUMOUR-INFILTRATING T CELLS

(71) Applicant: LYTIX BIOPHARMA AS, Tromso (NO)

(72) Inventors: Ketil André Camilio, Oslo (NO); Janne Merethe Nestvold, Oslo (NO); Baldur Sveinbjôrnsson, Oslo (NO); ØYstein Rekdal, Billingstad (NO)

(21) Appl. No.: 18/157,494

(22) Filed: Jan. 20, 2023

Related U.S. Application Data
(62) Division of application No. 16/070,803, filed on Jul. 18, 2018, now abandoned.

(30) Foreign Application Priority Data
Feb. 2, 2016 (GB) 1601868.1

(51) Int. Cl. C12N 5/0783; A61K 38/04; A61K 39/00; A61P 35/00; C07K 4/00; C07K 7/06; C07K 14/00

(52) U.S. Cl. CPC C12N 5/0638; A61K 38/04; A61K 39/4611; A61K 39/46449; A61K 2239/57; A61P 35/00; C07K 4/00; C07K 7/06; C07K 14/00

(57) ABSTRACT

Methods of generating populations of tumour-infiltrating T cells The present invention provides a method of generating a population of tumour-infiltrating T cells, said method comprising administering to a subject a positively charged amphipathic amino acid derivative, peptide or peptidomimetic which is able to lyse tumour cell membranes and then collecting a cellular sample from a tumour within said subject and separating T cells therefrom. The present invention further provides a method of generating a population of tumour-infiltrating T cells, said method comprising separating T cells from a cellular tumour sample taken from a subject treated with a positively charged amphipathic amino acid derivative, peptide or peptidomimetic which is able to lyse tumour cell membranes and optionally culturing said T cells. The present invention also provides the tumour-infiltrating T cells described above for use in treating tumour cells or preventing or reducing the growth, establishment, spread, or metastasis of a tumour.

### CROSS-REFERENCE TO RELATED APPLICATIONS

This application is a divisional of and claims priority to U.S. patent application Ser. No. 16/070,803 filed Jul. 18, 2018 (Publication No. 2019/0038670 published Feb. 7, 2019), which is a national phase filing of International Patent Application No. PCT/EP2017/052279 filed Feb. 2, 2017 (Publication No. WO 2107/134175 published Aug. 10, 2017), which claims priority to United Kingdom Patent Application No. 1601868.1 filed Feb. 2, 2016.

### REFERENCE TO A SEQUENCE LISTING

This application incorporates by reference the Sequence Listing submitted in Computer Readable Form as File 10sequencelisting.xml created on May 26, 2023 and containing 97,651 bytes.

The present invention relates to the field of cancer therapy and related investigations. In particular, the invention provides methods of generating T cell populations of scientific, diagnostic or therapeutic relevance.

The prevalence of cancer in human and animal populations and its role in mortality means there is a continuing need for new therapies to combat tumours and tumour cells. Elimination of a tumour, a reduction in its size, the disruption of its supporting vasculature, or reducing the number of cancer cells circulating in the blood or lymph systems may be beneficial in a variety of ways; e.g. by reducing pain or discomfort, preventing metastasis, facilitating operative intervention or prolonging life.

Cancer therapies which are designed to combat tumours or cells metastasising from tumours typically rely on a cytotoxic activity. That activity might be a cytotoxic effect an active agent has itself or it might be an effect employed indirectly by the active agent, e.g. the upregulation of the host immune response against a tumour. To a greater or lesser extent these therapies are selective for the target (tumours or tumour cells) rather than normal healthy, or at least non-cancerous, cells and tissues. Those therapies that are poorly selective are associated with serious side effects as normal cells are exposed to the cytotoxic activity that the active agent relies upon to exert its therapeutic effect.

Genetic and epigenetic alterations that are characteristic of cancers result in antigens that the immune system can recognise and use to differentiate between tumour cells and their healthy equivalents. In principle, this means that the immune system could be a powerful weapon in controlling tumours. However, the reality is that the immune system usually does not provide a strong response to tumour cells. It is of great therapeutic interest to manipulate and therefore harness the immune system in the fight against cancer (Mellman et al. Nature 2011, vol. 480, 480-489).

Various attempts have been made to help the immune system to fight tumours. One early approach involved a general stimulation of the immune system, e.g. through the administration of bacteria (live or killed) to elicit a general immune response which would also be directed against the tumour. This is also called nonspecific immunity.

Recent approaches aimed at helping the immune system specifically to recognise tumour-specific antigens involve administration of tumour-specific antigens, typically combined with an adjuvant (a substance which is known to cause or enhance an immune response) to the subject. Often not all the tumour-specific antigens have been identified, e.g. in breast cancer the known antigens are found in 20-30% of the total tumours. The use of tumour-specific vaccines, cancer vaccines, have so far met with limited success.

There remains a need for alternative methods for treating tumours and for alternative methods for inhibiting the growth or formation of secondary tumours.

It is recognised that the usual lack of a powerful immune response to tumour associated antigens (TAAs) is due to a combination of factors. T cells have a key role in the immune response, which is mediated through antigen recognition by the T cell receptor (TCR), and they coordinate a balance between co-stimulatory and inhibitory signals known as immune checkpoints (Pardoll, Nature 2012, vol. 12, 252-264). Inhibitory signals suppress the immune system which is important for maintenance of self-tolerance and to protect tissues from damage when the immune system is responding to pathogenic infection. However, immune suppression reduces what could otherwise be a helpful response by the body to the development of tumours. Cytokines, other stimulatory molecules such as CpG (stimulating dendritic cells), Toll-like receptor ligands and other molecular adjuvants enhance the immune response. Co-stimulatory interactions involving T cells directly can be enhanced using agonistic antibodies to receptors including OX40, CD28, CD27 and CD137. These are all “push”-type approaches to cancer immunotherapy.

Complementary ‘pull’ therapies may block or deplete inhibitory cells or molecules and include the use of antagonistic antibodies against what are known as immune checkpoints. Immune checkpoints include CTLA-4 and PD-1 and antibodies against these are known in the art; ipilimumab was the first FDA-approved anti-immune checkpoint antibody licensed for the treatment of metastatic melanoma and this blocks cytotoxic T-lymphocyte antigen 4 (CTLA-4) (Naidoo et al. British Journal of Cancer (2014) 111, 2214-2219). There are other agents which would be considered classic chemotherapeutics which can reduce immune suppression at sub-cytotoxic doses, these include cyclophosphamide and doxorubicin.

T cells are central to the immune response to cancers and there is interest in the field in using tumour infiltrating lymphocytes (TILs) in the treatment and understanding of cancer. Through their T cell receptors (TCRs), T cells are reactive to specific antigens within a tumour. Tumour cells carry genetic mutations, many of which contribute directly or indirectly to malignancy. A mutation in an expressed sequence will typically result in a neoantigen, an antigen that is not known to the immune system and thus recognised as foreign and able to elicit an immune response.

Neoantigens and their responsible mutations vary between tumour types and even between patients with the same tumour; the extent of genetic mutations is referred to as the mutational load. A tumour with a large number of mutations is expected to be more immunogenic and may be considered immunogenically “hot”. A tumour with a small mutational load will typically be less immunogenic and thus “cold”. A tumour may also appear “cold” if the immune system is unable to access its neoantigens. Just as there is interest in the therapeutic potential of TILs, so there is interest in understanding and utilising neoantigens in cancer immunotherapy (Schumacher and Schreiber, Science 2015, vol 348, issue 6230, pp 69-74).

Thus there is a relationship between the TIL population and the accessible neoantigens in a tumour, with many TILs specific to particular neoantigens. This opens up several avenues of therapy, such as adoptive T cell therapy, where a tumour sample is taken and T cells isolated therefrom and then clones from within this repertoire of TILs are cultured in vitro and then returned to the body to boost the natural immune response. Such techniques, which rely on collection of TILs, are described by Linnemann et al in Immunological Reviews 2014, Vol 257:77-82.

The present inventors have established that some peptides, known to lyse tumour cells through disturbing and permeabilizing the cell membrane, are also highly effective at attacking organelles such as mitochondria and lysosomes and can cause lysis thereof. This may be achieved at low concentrations which do not cause direct lysis of the cell membranes, although loss of cell membrane integrity is seen eventually even on administration of low doses. At higher doses, these molecules can cause lysis of the cell membrane and then of the membranes of organelles. It is believed that this results in the release of a more complete repertoire of TAAs.

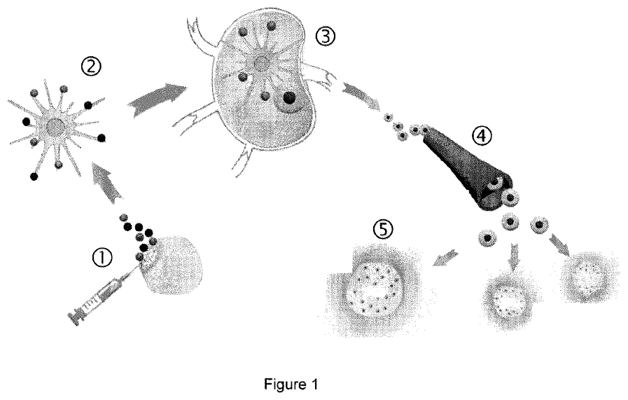

This disruption of the organelle membrane also results in the release of agents therefrom which have a potent immunostimulatory function, such agents are generally known as DAMPs (Damage-associated molecular pattern molecules) and include ATP, Cytochrome C, mitochondrial CpG DNA sequences, mitochondrial formyl peptides, cathepsins (from lysosomes) and HMGB1 (from the nucleus). This effect can enhance the T cell response to TAAs, including neoantigens. For example, DAMPs play a key role in activating, recruiting and the subsequent maturation of antigen presenting cells. As shown in FIG. 1, the maturation of antigen presenting cells and the migration of such cells to the lymph node is the prerequisite for the presentation of the tumour antigens to the T cells.

The present inventors now postulate that analysis of the TIL population which may be isolated from a tumour which has been treated with a membrane acting lytic compound is able to identify useful neoantigens within the tumour. Thus a TIL population isolated from a tumour which has been treated with a membrane acting lytic compound is surprisingly much more useful than TIL populations obtained from untreated tumours. This proposed utility is based, inter alia, on the observation, described in the Examples herein, that the TIL population obtained from a tumour treated with a membrane acting lytic compound has increased clonality as compared to a TIL population from an untreated tumour. Clonality is high when the relative abundance of T cells across the different clonotypes is not evenly distributed, i.e. certain clonotypes dominate the total TIL population. It is believed that clonotypes which are well represented in the TIL population will be reactive to neoantigens which have been made more easily available as a result of the tumour treatment and lysis of tumour cells (see FIG. 1 in this regard). Such clonotypes may be used, for example, in autologous T cell therapy or for identification of neoantigens which may be used in vaccination therapy or in the generation of neoantigen libraries.

Thus, in a first aspect, the present invention provides a method of generating a population of tumour-infiltrating T cells, said method comprising administering to a subject a positively charged amphipathic amino acid derivative, peptide or peptidomimetic which is able to lyse tumour cell membranes and then collecting a cellular sample from a tumour within said subject and separating T cells therefrom.

It will be understood that separation of T cells or of individual clonotypes may include isolation or partial isolation from other cell types or cell debris. The resulting populations may be 80, 90, 95, 98 or 99% pure in terms of cell type.

In a further aspect the present invention provides a method of generating a population of tumour-infiltrating T cells, said method comprising separating T cells from a cellular tumour sample taken from a subject (who has been) treated with a positively charged amphipathic amino acid derivative, peptide or peptidomimetic which is able to lyse tumour cell membranes and optionally culturing said T cells.

The collected T cell population typically comprises a plurality of T cell clonotypes. Optionally the resulting T cell population is then cultured to maintain or expand the population. Optionally the T cell population may be enriched for certain clonotypes and/or fractionated to separate clonotypes into sub-populations comprising 1 or more clonotypes, e.g. 1-10, 1-5 or 1, 2 or 3 clonotypes per sub-population.

The cellular tumour sample may comprise all or part of a solid tumour lesion and will typically comprise tumour cells as well as TILs, of which some will be T cells. Methods of harvesting T cells from a tumour sample, i.e. of separating T cells from such a sample, are known in the art.

The generated population of T cells may be analysed, for example to assess clonality, as described in the present Examples. The T cells may be analysed to investigate properties, e.g. binding affinity or sequence of their T cell receptors (TCRs).

Without wishing to be bound by theory, it is believed that some of the clonotypes within the T cell population will be reactive to (specific for) tumour neoantigens, specifically that some of the most highly represented clones will be reactive to tumour neoantigens. Thus methods of the invention may further comprise a step of analysing the generated T cells in order to identify their corresponding tumour neoantigen.

In a further embodiment, the methods of generating a population of tumour-infiltrating T cells discussed above comprise a further step of expanding the T cells ex vivo. This expansion can be carried out using standard cell culture methods known in the art. In a further aspect, the present invention provides the isolated T cells obtained by the methods defined above.

The tumour-infiltrating T cells that are generated from the methods defined above may be used therapeutically as part of an adoptive cell transfer therapy strategy in order to treat a patient suffering from a tumour. Thus, the methods of the invention may comprise a further step of administering the generated and optionally expanded T cells to a subject.

In a further aspect, the present invention provides a population of T cells defined above for use in treating tumour cells or preventing or reducing the growth, establishment, spread or metastasis of a tumour in a subject.

Thus, for example, the present invention provides a population of expanded T cells generated from the ex vivo methods defined above for use in treating tumour cells or preventing or reducing the growth, establishment, spread or metastasis of a tumour in a subject.

Such a strategy is known in the art, as described in Schumacher & Schreiber, Science 2015, 348: 69-74. A small selection of clonotypes, e.g. 1 to 20, 1 to 15, 1 to 10 or 1 to 5 clonotypes are preferably administered.

Alternatively viewed, the present invention provides a method of treating tumour cells or preventing or reducing the growth, establishment spread, or metastasis of a tumour, which method comprises administering a therapeutically effective amount of T cells generated from the methods defined above to a subject in need thereof.

Alternatively viewed, the present invention provides the use of T cells generated from the methods defined above in the manufacture of a medicament for treating tumour cells or preventing or reducing the growth, establishment spread, or metastasis of a tumour in a subject.

The subject treated may be different from the subject that is administered with the amphipathic amino acid derivative, peptide or peptidomimetic, but preferably the subject is the same. Such a strategy would increase the sensitivity of the subject's immune system towards the antigens or neoantigens present on the surface of the tumour and therefore increase the likelihood that the immune system clears the tumour tissue. This strategy would also increase the sensitivity of the immune system towards subsequent metastases that may develop.

The T cells may also be used to identify the tumour-specific antigens or neoantigens that bind to the T cells. Thus, the methods of the invention may comprise a further step of identifying tumour-specific antigens or neoantigens. This method may be carried out after the T cells are expanded ex vivo, or alternatively this method may be carried out directly on the T cells that are generated in vivo. In a further aspect, the present invention provides the use of the T cells generated from the methods defined above in identifying tumour-specific antigens or neoantigens capable of binding to said T cells. Methods of identifying such neoantigens are known in the art and are described in Linnemann C et al., Immunol. Rev. 2014, 257: 72-82. Briefly, neoantigens may be identified through comparison of the cancer exome with the healthy patient exome in order to determine mutations, then synthesising mutated peptides based on the mutations presented in the cancer exome, then screening the mutated peptides against the T cells that have been generated. Alternatively, antigens or neoantigens may be identified through sequencing of the receptors of the T cell population in order to identify peptide motifs that would likely bind to the T cell receptors. Methods involving TCR sequencing of abundant (e.g. top 10, 20 or 30) clonotypes generated by the methods of the invention are preferred further aspects of the invention.

In a further aspect, the present invention provides the antigens or neoantigens obtained by the methods defined above.

Preferably, the method of identifying tumour-specific antigens or neoantigens may comprise a further step of synthesising the identified antigen or neoantigen and optionally administering the antigen or neoantigen to a subject in need, thereby treating tumour cells or preventing or reducing the growth, establishment spread, or metastasis of a tumour. In a further aspect, the present invention provides an antigen or neoantigen identified using the method defined above for use in treating tumour cells or preventing or reducing the growth, establishment spread, or metastasis of a tumour. Administration of antigens or neoantigens would again prime the subject's immune system towards the same antigens or neoantigens present on the surface of the tumour and therefore increase the likelihood that the immune system clears the tumour tissue. This strategy would also increase the sensitivity of the immune system towards subsequent metastases that may develop.

The antigens or neoantigens may be administered not only to the subject that is administered with the amphipathic amino acid derivative, peptide or peptidomimetic, but also to other subjects suffering from a tumour. However, many antigens and neoantigens are specific to an individual patient due to interpatient tumour heterogeneity, and so preferably the antigen or neoantigen is administered to the subject that is initially administered with the amphipathic amino acid derivative, peptide or peptidomimetic.

The identified antigen or neoantigen would act as a vaccine, stimulating an immune response that is specific to a particular tumour. Thus, the antigens or neoantigens may be modified in order to make them more immunogenic. For example, the antigen or neoantigen may be bound to a major histocompatibility complex protein in order to increase immunogenicity of the antigen or neoantigen. In addition, the antigen or the neoantigens, or the expanded T cells discussed above, may be administered with vaccine adjuvants that again increase immunogenicity.

Alternatively viewed, the present invention provides a method of treating tumour cells or preventing or reducing the growth, establishment spread, or metastasis of a tumour, which method comprises administration of a therapeutically effective amount of an antigen or a neoantigen identified using the method defined above.

Alternatively viewed, the present invention provides the use of the antigens or neoantigens identified using the method defined above in the manufacture of a medicament for treating tumour cells or preventing or reducing the growth, establishment spread, or metastasis of a tumour in a subject.

The above methods of treatment preferably comprise co-administration with a checkpoint inhibitor.

The above methods may be used to generate a library of T cell clones (clonotypes) or neoantigens specific to an individual or to a particular tumour type.

The methods of the invention involve generation of a T cell population and may further comprise identification and/or isolation of one or more T cell clonotypes from said T cell population. Preferably methods comprise identification and/or isolation of a plurality of T cell clonotypes from said T cell population.

The unique combination of properties exhibited by the membrane active molecules defined herein result in a particularly useful T cell population. The molecules are preferably able to cause loss of integrity of intracellular membranes, e.g. mitochondrial or lysosomal membranes or nuclear membranes, mitochondrial and lysosomal membranes are preferred. This loss of integrity is sufficient to cause a release of at least some of the content of the organelle and may include disintegration of the membrane. In this process antigens are made available for recognition by dendritic cells which trigger the maturation of specific T cells which are able to bind to those antigens. This results in increased infiltration of the tumour by T cells which are specific for these new antigens. The T cell response to neoantigens is strong and means the infiltrating T cells include a large proportion of clonotypes which are able to recognise neoantigens within the tumour. For a tumour which was previously only weakly immunogenic, this disruption of cellular and, in particular, intracellular membranes, results in the release of a broad range of TAAs, in particular previously ‘hidden’ tumour neoantigens. In turn this results in a greater T cell response.

Methods of testing for loss of integrity of intracellular membranes are known in the art, e.g. testing for release of cytochrome c, and suitable methods are described in the Examples. Methods of testing for cell lysis are also known in the art and described in the Examples, including the use of transmission electron microscopy.

This exceptionally broad range of released and recognisable TAAs essentially makes the tumour much more immunogenic and thus detectable by the immune system which is then able to contribute to regression of the initially treated and other tumours present in the body.

Thus, in a further aspect, the present invention provides an amino acid derivative, peptide or peptidomimetic as defined herein of use in generating, in vivo, a population of tumour-infiltrating T cells for use in the treatment of a tumour. The amino acid, peptide or peptidomimetic is administered to a subject with a tumour, preferably administration is intratumoural. The population of T cells generated preferably includes clonotypes to antigens not previously recognised (to a therapeutically relevant extent) by the subject's immune system.

The TIL population generated after tumour cell lysis may be modified in order to make it more immunogenic, i.e. more likely to activate an adaptive immune response upon detecting a tumour-specific antigen. Methods of modifying TIL populations are known in the art. For example, research has been carried out into genetically modifying the T cell receptor (TCR) that can alter T-cell specificity. This is carried out through identifying TCR α and β chains that are specific for the tumour antigen of interest, isolating and cloning the corresponding nucleic acid sequences into transduction vectors and the transduction of the T cells. This technique allows for in vitro modifications of the α and β chains in order to further improve the interaction between the TCR and the antigen (avidity).

An alternative strategy is to form chimeric antigen receptor (CAR) T cells. These CAR T cells combine both antibody-like recognition with T-cell activating function. CARs are composed of an antigen-binding domain, typically derived from a monoclonal antibody, a transmembrane domain that anchors the CAR to the T cell and one or more intracellular signalling domains that induce persistence, trafficking and effector function once a tumour antigen has bound to the CAR. The intracellular signalling domains lead to the long-lasting activation of the T cells. The concepts of improving the immunogenicity of a TIL population described above are known in the art, as reviewed in Sharpe & Mount in Dis. Model Mech. 2015, 8: 337-50.

Treatment with a membrane-acting lytic compound can lead to TILs that identify useful neoantigens within the tumour; it is also recognised that these TILs can aid the development of useful TCRs and CARs. In particular, analysis of the resulting TCR of the TILs, using for example x-ray crystallography, may be carried out in order to determine the structure of the antigen-binding region, and this structural analysis can then be used in order to develop new TCRs and CARs that mimic, or even improve upon antigen binding. Alternatively, such new TCRs and CARs can be developed through analysing the structure of the neoantigen. These TCRs and CARs can be modified in order to improve immune cell activation as discussed above. These new TCRs and CARs can then be genetically introduced into T cells (that may or may not be tumour-specific) so that the cells express them on the cell membrane. These new TCRs and CARs can also be genetically introduced into natural killer (NK) cells. These T cells may be the same as the TILs used to identify the useful neoantigens.

These genetically modified T cells or NK cells may then be administered to a patient in order to treat a tumour. The genetically introduced TCRs or CARs may have been derived from the same patient that is administered with the genetically modified T cells or NK cells for treatment, or alternatively the patient may be different (preferably the patient is the same).

The molecules of use in the methods of the invention are amphipathic in that they have a hydrophilic, i.e. cationic part or parts, and a hydrophobic part or parts. Thus the molecules are attracted to the negative charge of phospholipid membranes and able to interact with the fatty chains of the lipid membranes with their hydrophobic group(s).

Amphipathic molecules with an ability to lyse phospholipid membranes, cellular or intracellular, are known in the art and may be termed lytic molecules. Lysis includes destabilisation of the membrane such that it loses its functional integrity and normal ability to compartmentalise, e.g. maintain osmotic pressure or a pH gradient or other concentration gradient. Typically lysis will result in partial or complete disintegration of the lipid bi-layer, which may be seen with a microscope and include a loss of cytoplasm and loss of gross cell wall structure.

The amino acids which may be used are derivatives as they are not naturally occurring amino acids and typically include modifications to the standard amino acid structure, e.g. a modified carboxyl group.

The molecules of use according to the invention include the group of peptides commonly known as Cationic antimicrobial peptides (CAPs). These are positively charged amphipathic peptides and peptides of this type are found in many species and form part of the innate immune system. The CAP Lactoferricin (LfcinB) is a 25 amino acid peptide which has been shown to have an effect on mitochondria (Eliasen et al. Int. J. Cancer (2006) 119, 493-450). It has also been found that the much smaller peptide LTX-315, a 9 amino acid peptide (of the type described in WO 2010/060497), also targets the mitochondria.

Each molecule preferably contains at least two cyclic groups. The cyclic group is preferably a 5 or 6 membered ring (although larger rings, e.g. rings of 7, 8, 9 or 10 non-hydrogen atoms, can be used) which may be aliphatic or aromatic, preferably aromatic, and may be substituted, substituting groups may include heteroatoms such as oxygen, nitrogen, sulphur or a halogen, in particular fluorine, bromine or chlorine. Preferred substituting groups include C₁-C₄ alkyl (especially t-butyl), methoxy, fluoro and fluoromethyl groups. The cyclic group may be homo- or heterocyclic, preferably a homocyclic ring of carbon atoms. The cyclic groups may be connected or fused, preferably fused. Particularly preferred side-chains comprise a naphthalene or an indole group. A further preferred group of lipophilic side chains have a single substituted or unsubstituted cyclic group, preferably a phenyl or cyclohexyl group.

Single amino acid derivatives may be employed provided they have the necessary amphipathicity. They will carry at least one, preferably at least 2 positive charges and to exhibit adequate cationicity will typically have a modified C terminus, e.g. amidated or esterified, possibly with addition of a lipophilic group of 6 or more non-hydrogen atoms. A single amino acid derivative will need to contain lipophilic group(s) which are able to perturb phospholipid membranes, e.g. a single group of 10 or more or 12 or more non-hydrogen atoms such as tri-butyl tryptophan. The amino acid may include 2 or more lipophilic groups, each of at least 6 non-hydrogen atoms. Preferred amino acid derivatives are β amino acids which are disubstituted, as described in further detail below.

Preferred peptides may consist of 2 to 25 (preferably 2 to 20 or 2 to 15, more usually 6 to 10) amino acids and have a net positive charge at pH 7.2-7.6. Preferably (i) 2 or more (e.g. 2 or 3 to 15 or 18) of the amino acids have a cationic side chain, and (ii) one or more (e.g. 1 or 2 to 6) amino acids have a lipophilic side chain, e.g. incorporating at least one cyclic group and at least 7 non-hydrogen atoms.

Peptides typically comprise one or more amino acids having a lipophilic side chain incorporating at least one cyclic group and at least 7 non-hydrogen atoms (including the cyclic group). Preferably the peptides comprise 1 to 6, more preferably 1 to 4, e.g. 1, 2 or 3 lipophilic side chains. All such amino acids and side chains thereof may conveniently be referred to as “bulky and lipophilic” amino acids/side chains. Preferably, the side chain contains at least 8, more preferably at least 10 non-hydrogen atoms. Preferred lipophilic side chains incorporate two or three cyclic groups, preferably two cyclic groups, as defined above.

Of the genetically coded amino acids phenylalanine (7 non hydrogen atoms), tryptophan (10 non hydrogen atoms) and tyrosine (8 non hydrogen atoms) are suitable bulky and lipophilic amino acids. Tryptophan, because of its two fused ring structure and additional bulk is particularly preferred. Non-genetic amino acids, which may be naturally occurring, and tryptophan, phenylalanine and tyrosine analogues and amino acids which have been modified to incorporate a lipophilic group as defined above may also be used, e.g. tryptophan residues which have been substituted at the 1-, 2-, 5- and/or 7-position of the indole ring, positions 1- or 2-being preferred e.g. 5′ hydroxy tryptophan. A variety of other amino acid derivatives having a bulky and lipophilic character are known to the man skilled in the art.

Preferred non-genetically coded bulky and lipophilic amino acids include adamantylalanine; 3-benzothienylalanine; biphenylalanine, e.g. 4,4′-biphenylalanine; diphenylalanine, e.g. 3,3-diphenylalanine; a biphenylalanine derivative, e.g. Bip (4-(2-Naphthyl)), Bip (4-(1-Naphthyl)), Bip (4-n-Bu), Bip (4-Ph) or Bip (4-T-Bu) or Phe (4-(2′-naphthyl)), Phe (4-(1′-naphthyl)), Phe (4-n-butylphenyl), Phe (4-4′-biphenyl) or Phe (4′-t-butylphenyl); homophenylalanine; 2,6-dichlorobenzyltyrosine, cyclohexyltyrosine; 7-benzyloxytryptophan; tributyl tryptophan (Tbt), e.g. tri-tert.-butyltryptophan; homotryptophan; 3-(-anthracenyl)-L-alanine; L-p-iso-propylphenylalanine; thyroxine; 3,3′,5-triiodo-L-thyronine; triiodo-tyrosine; 2-amino-3-(anthracen-9-yl) propanoic acid; 2-amino-3-(naphthalen-2-yl) propanoic acid; 2-amino-3-(naphthalen-1-yl) propanoic acid; 2-amino-3-[1,1′:4′,1″-terphenyl-4-yl]-propionic acid; 2-amino-3-(2,5,7-tri-tert-butyl-1H-indol-3-yl) propanoic acid; 2-amino-3-[1,1′:3′,1″-terphenyl-4-yl]-propionic acid; 2-amino-3-[1,1′:2′,1″-terphenyl-4-yl]-propionic acid; 2-amino-3-(4-naphthalen-2-yl-phenyl)-propionic acid; 2-amino-3-(4′-butylbiphenyl-4-yl) propanoic acid; 2-amino-3-[1,1′:3′,1″-terphenyl-5′-yl]-propionic acid; and 2-amino-3-(4-(2,2-diphenylethyl) phenyl) propanoic acid.

Preferred peptides include at least one, e.g. 1-4, typically 1 or 2 non-genetically coded amino acids, e.g. biphenylalanine or diphenylalanine.

A lipophilic molecule is one which associates with its own kind in an aqueous solution, not necessarily because the interactions between the lipophilic molecules are stronger than between the lipophilic molecule and water but because interactions between a lipophilic molecule and water would destroy the much stronger interactions between the water molecules themselves. It is therefore preferable that the lipophilic side chain should not contain many polar functional groups e.g. no more than 4, preferably 2 or less, e.g. one or none. Such groups would increase the binding interaction with the aqueous surroundings and hence lower the lipophilicity of the molecule. The slight polarity of a side-chain like tryptophan's is tolerated and indeed, tryptophan is a preferred bulky and lipophilic amino acid found in the second peptide.

Standard chemical protecting groups when attached to an amino acid side chain can provide suitable bulky and lipophilic side chains. Suitable amino acid protecting groups are well known in the art and include Pmc (2,2,5,7,8- pentamethylchroman-6-sulphonyl), Mtr (4-methoxy-2,3,6-trimethylbenzenesulfonyl) and Pbf (2,2,4,6,7-pentamethyldihydrobenzofuransulfonyl), which may conveniently increase the bulk and lipophilicity of aromatic amino acids, e.g. phenylalanine, tryptophan and tyrosine. Also, the tert.-butyl group is a common protecting group for a wide range of amino acids and is capable of providing a bulky and lipophilic group to amino acid side chains, particularly when modifying aromatic side chains. The Z-group (carboxybenzyl) is a further protecting group which can be used to provide a bulky and lipophilic group.

A further lipophilic group incorporating at least one cyclic group and at least 7 non-hydrogen atoms may be present as an N or C-terminal modification and the above discussion of preferred bulky and lipophilic groups applies, mutatis mutandis, to this group.

N-terminal modifications providing the further bulky and lipophilic group may be attached directly to the N-terminal amine by any convenient means to form a mono-, di- and possibly cationic trialkylated N-terminal amine. Alternatively, the further bulky and lipophilic group (“R” in the following paragraphs) may be attached via a linking moiety e.g. a carbonyl group (RCO) e.g. adamantyl or benzyl, carbamate (ROCO), or a linker which forms urea (RNHCO) or (R₂NCO) or by a linker which forms a sulfonamide, boronamide or phosphonamide. Sulfonamide forming linkers may be particularly useful when a more stable peptide is required.

A bulky and lipophilic group as defined above may also be provided by a C-terminal modifying group. Bulky and lipophilic groups may be attached directly to the C-terminal carboxy group to form a ketone. Alternatively, bulky and lipophilic groups may be attached via a linking moiety, e.g. (OR) which forms an ester at the C-terminus, (NH—R) or (NR₂, wherein the two R groups needs not be the same) which form primary and secondary amide groups respectively at the C-terminus or groups (B-(OR)₂) which form boronic esters or phosphorous analogues. Dae (diaminoethyl) is a further linking moiety which may be used to attach a bulky and lipophilic group, e.g. carbobenzoxy (Z) to the C-terminus.

It will be appreciated that the number of cationic residues will likely be proportional to the length of the peptide, e.g. ⅓ to ¾ of the residues are cationic. Likewise, 1/4 to 2/3 of the residues are lipophilic (preferably with 7 or more non-hydrogen atoms).

In certain embodiments the peptide may contain 3, 4, 5, 6, 7, 8, 9, 10, 11, 12, 13, 14, 15, 16, 17, 18, or 19 amino acids with a cationic side chain. For ease of reference, these amino acids will be referred to in the following sections as “cationic residues”.

In further embodiments where the peptide consists of 8, 9, 10 or 11 amino acids, it may comprise 3 to 10, e.g. 4 to 9, 5 to 8, 6 to 7 or 5 cationic residues. In still further embodiments where the peptide consists of 4, 5, 6 or 7 amino acids, it may comprise 2 to 6, e.g. 3 or 4 cationic residues.

In certain embodiments the peptide may contain 3, 4, 5, 6, 7, 8, 9, 10, 11, 12, 13, 14, 15, 16, 17 or 18 bulky and lipophilic amino acids.

In certain embodiments where the peptide consists of 16, 17, 18, 19 or 20 amino acids, it may comprise 3 to 17, 4 to 16, 5 to 15, 6 to 14, 7 to 13, 8 to 12, 9 to 11 or 10 bulky and lipophilic residues. In further embodiments where the peptide consists of 8, 9, 10 or 11 amino acids, it may comprise 3 to 8, e.g. 4 to 7, bulky and lipophilic residues. In still further embodiments where the peptide consists of 4, 5, 6 or 7 amino acids, it may comprise 2 to 5, e.g. 3 or 4 bulky and lipophilic residues.

The arrangement of the bulky and lipophilic and cationic residues and the further bulky and lipophilic group in the peptide is not of paramount importance to the functioning of the invention.

By “amino acid with a cationic side chain” it is meant an amino acid that has a side chain that has a net positive charge at the intracellular pH of a tumour cell, e.g. around pH 7.4. Of the genetically coded amino acids this would include lysine and arginine but any non-genetically coded or modified amino acid carrying such a net positive charge on its side chain may be used, e.g. those amino acids carrying a side-chain with a guanidino group or an amine group or another cationic moiety, e.g. derivatives of lysine, and arginine in which any hydrogen in the side chain, except the protonating hydrogen, is substituted with a halogen atom, e.g. fluorine, chlorine or bromine, or a linear, branched aliphatic unsaturated or saturated C₁-C₄ alkyl or alkoxy group, e.g. methyl, ethyl, propyl, iso-propyl, butyl, iso-butyl, sec-butyl, tert-butyl, ethylene, propylene, butylene, hydroxy, methoxy, ethyloxy, propyloxy, iso-propyloxy, butyloxy group, iso-butyloxy, sec-butyloxy, tert-butyloxy or halogen substituted versions thereof.

Suitable non-genetically coded amino acids with cationic side chains include homolysine, ornithine, diaminobutyric acid, diaminopimelic acid, diaminopropionic acid and homoarginine as well as trimethylysine and trimethylornithine, 4-aminopiperidine-4-carboxylic acid, 4-amino-1-carbamimidoylpiperidine-4-carboxylic acid and 4-guanidinophenylalanine.

Amino acids, with the exception of glycine, may exist as two or more stereoisomers. In particular the a-carbon of an amino acid other than glycine is a chiral centre and so gives rise to two enantiomeric forms of each amino acid. These forms are often referred to as D and L forms, e.g. D-alanine and L-alanine. Amino acids with further chiral centres will exist in four or more possible stereoisomers, e.g. threonine has two chiral centres and so may exist in one of four stereoisomeric forms. Any stereoisomeric form of an amino acid may be used in the molecules of the invention. For the purposes of describing the present invention, where the term “non-genetically encoded” is applied to amino acids, this does not include the D forms of amino acids that occur in nature in the L form.

Preferably, the positively charged amphipathic amino acid derivative, peptide or peptidomimetic of the present invention is further defined as set out in the sections below.

### Lytic Nonapeptides

As described in patent publication WO2010/060497, the lytic peptide or peptidomimetic may have the following characteristics:
a) consisting of 9 amino acids in a linear arrangement;
b) of those 9 amino acids, 5 are cationic and 4 have a lipophilic R group;
c) at least one of said 9 amino acids is a non-genetically coded amino acid or a modified derivative of a genetically coded amino acid; and optionally
d) the lipophilic and cationic residues are arranged such that there are no more than two of either type of residue adjacent to one another; and further optionally
e) the molecule comprises two pairs of adjacent cationic amino acids and one or two pairs of adjacent lipophilic residues.

The cationic amino acids, which may be the same or different, are preferably lysine or arginine but may be histidine or any non-genetically coded or modified amino acid carrying a positive charge at pH 7.0.

Suitable non-genetically coded cationic amino acids and modified cationic amino acids include analogues of lysine, arginine and histidine such as homolysine, ornithine, diaminobutyric acid, diaminopimelic acid, diaminopropionic acid and homoarginine as well as trimethylysine and trimethylornithine, 4-aminopiperidine-4-carboxylic acid, 4-amino-1-carbamimidoylpiperidine-4-carboxylic acid and 4-guanidinophenylalanine.

The lipophilic amino acids (i.e. amino acids with a lipophilic R group), which may be the same or different, all possess an R group with at least 7, preferably at least 8 or 9, more preferably at least 10 non-hydrogen atoms. An amino acid with a lipophilic R group is referred to herein as a lipophilic amino acid. Typically the lipophilic R group has at least one, preferably two cyclic groups, which may be fused or connected.

The lipophilic R group may contain hetero atoms such as O, N or S but typically there is no more than one heteroatom, preferably it is nitrogen. This R group will preferably have no more than 2 polar groups, more preferably none or one, most preferably none.

Tryptophan is a preferred lipophilic amino acid and the molecules preferably comprise 1 to 3, more preferably 2 or 3, most preferably 3 tryptophan residues. Further genetically coded lipophilic amino acids which may be incorporated are phenylalanine and tyrosine.

Preferably one of the lipophilic amino acids is a non-genetically coded amino acid. Most preferably the molecule consists of 3 genetically coded lipophilic amino acids, 5 genetically coded cationic amino acids and 1 non-genetically coded lipophilic amino acid. In this context, a D amino acid, while not strictly genetically coded, is not considered to be a “non-genetically coded amino acid”, which should be structurally, not just stereospecifically, different from the 20 genetically coded L amino acids. The molecules of the invention may have some or all of the amino acids present in the D form, preferably however all amino acids are in the L form.

When the molecules include a non-genetically coded lipophilic amino acid (or amino acid derivative), the R group of that amino acid preferably contains no more than 35 non-hydrogen atoms, more preferably no more than 30, most preferably no more than 25 non-hydrogen atoms.

Preferred non-genetically coded amino acids include: 2-amino-3-(biphenyl-4-yl)propanoic acid (biphenylalanine), 2-amino-3,3-diphenylpropanoic acid (diphenylalanine), 2-amino-3-(anthracen-9-yl)propanoic acid, 2-amino-3-(naphthalen-2-yl)propanoic acid, 2-amino-3-(naphthalen-1-yl)propanoic acid, 2-amino-3-[1,1′:4′,1″-terphenyl-4-yl]-propionic acid, 2-amino-3-(2,5,7-tri-tert-butyl-1H-indol-3-yl)propanoic acid, 2-amino-3-[1,1′:3′,1″-terphenyl-4-yl]-propionic acid, 2-amino-3-[1,1′:2′,1″-terphenyl-4-yl]-propionic acid, 2-amino-3-(4-naphthalen-2-yl-phenyl)-propionic acid, 2-amino-3-(4′-butylbiphenyl-4-yl)propanoic acid, 2-amino-3-[1,1′:3′,1″-terphenyl-5′-yl]-propionic acid and 2-amino-3-(4-(2,2-diphenylethyl)phenyl)propanoic acid.

In a preferred embodiment the compounds of the invention have one of formulae I to V listed below, in which C represents a cationic amino acid as defined above and L represents a lipophilic amino acid as defined above. The amino acids being covalently linked, preferably by peptide bonds resulting in a true peptide or by other linkages resulting in a peptidomimetic. The free amino or carboxy terminals of these molecules may be modified, the carboxy terminus is preferably modified to remove the negative charge, most preferably the carboxy terminus is amidated, this amide group may be substituted.

(I)
(SEQ ID NO: 1)
CCLLCCLLC
(II)
(SEQ ID NO: 2)
LCCLLCCLC
(III)
(SEQ ID NO: 3)
CLLCCLLCC
(IV)
(SEQ ID NO: 4)
CCLLCLLCC
(V)
(SEQ ID NO: 5)
CLCCLLCCL

β and γ amino acids as well as a amino acids are included within the term ‘amino acids’, as are N-substituted glycines. The compounds of the invention include beta peptides and depsipeptides.

As discussed above, the compounds of the invention incorporate at least one, and preferably one, non-genetically coded amino acid. When this residue is denoted L′, preferred compounds are represented by the following formulae:

(I′)
(SEQ ID NO: 6)
CCL′LCCLLC
(I″)
(SEQ ID NO: 7)
CCLLCCLL′C
(I″′)
(SEQ ID NO: 8)
CCLL′CCLLC
(II′)
(SEQ ID NO: 9)
LCCLL′CCLC

Particularly preferred are peptides of formula I and II, and of these, peptides of formula I″ are especially preferred.

The following peptides as presented in Table 1 are most preferred.

TABLE 1

| | SEQ | |
|---|---|---|
| | ID | |
| Name | NO | Sequence |
| LTX-301 | 10 | Dip-K-K-W-W-K-K-W-K-NH₂ |
| LTX-302 | 11 | W-K-K-W-Dip-K-K-W-K-NH₂ |
| LTX-303 | 12 | W-K-K-W-W-K-K-Dip-K-NH₂ |
| LTX-304 | 13 | Bip-K-K-W-W-K-K-W-K-NH₂ |
| LTX-305 | 14 | W-K-K-Bip-W-K-K-W-K-NH₂ |
| LTX-306 | 15 | w-k-k-w-dip-k-k-w-k-NH₂ |
| LTX-307 | 16 | K-K-W-Dip-K-K-W-W-K-NH₂ |
| LTX-308 | 17 | k-k-W-Dip-k-k-W-W-k-NH₂ |
| LTX-309 | 18 | K-K-W-Dip-K-K-W-Dip-K-NH₂ |
| LTX-310 | 19 | K-K-W-Bip-K-K-W-W-K-NH₂ |
| LTX-312 | 20 | K-Bip-K-K-W-W-K-K-W-NH₂ |
| LTX-313 | 21 | K-K-Bip-W-K-K-W-W-K-NH₂ |
| LTX-314 | 22 | K-K-W-W-K-K-Dip-W-K-NH₂ |
| LTX-315 | 23 | K-K-W-W-K-K-W-Dip-K-NH₂ |
| LTX-316 | 24 | K-W-Dip-K-K-W-W-K-K-NH₂ |
| LTX-317 | 25 | K-K-W-W-K-W-Dip-K-K-NH₂ |
| LTX-318 | 26 | Orn-Orn-W-Dip-Orn-Orn-W-W-Orn-NH₂ |
| LTX-319 | 27 | Dap-Dap-W-Dip-Dap-Dap-W-W-Dap-NH₂ |
| LTX-320 | 28 | R-R-W-Dip-R-R-W-W-R-NH₂ |
| LTX-321 | 29 | K-W-W-K-K-Dip-W-K-K-NH₂ |
| LTX-323 | 30 | K-Dip-K-K-W-W-K-K-W-NH₂ |
| LTX-324 | 31 | K-K-Dip-W-K-K-W-W-K-NH₂ |
| LTX-325 | 32 | k-w-w-k-k-dip-w-k-k-NH₂ |
| LTX-326 | 33 | R-R-Bip-W-R-R-W-W-R-NH₂ |
| LTX-327 | 34 | R-R-Dip-W-R-R-W-W-R-NH₂ |
| LTX-329 | 35 | k-k-bip-w-k-k-w-w-k-NH₂ |
| LTX-331 | 36 | k-k-Bip-w-k-k-w-w-k-NH₂ |
| LTX-332 | 37 | K-K-bip-W-K-K-W-W-K-NH₂ |
| LTX-333 | 38 | Dab-Dab-W-Dip-Dab-Dab-W-W-Dab-NH₂ |
| LTX-334 | 39 | K-K-W-1-Nal-K-K-W-W-K-NH₂ |
| LTX-335 | 40 | K-K-W-2-Nal-K-K-W-W-K-NH₂ |
| LTX-336 | 41 | K-K-W-Ath-K-K-W-W-K-NH₂ |
| LTX-338 | 42 | K-K-W-Phe(4-4′Bip)-K-K-W-W-K-NH₂ |

In which:
the standard single letter code is used for the genetically coded amino acids
lower case denotes D amino acids
Dip is diphenylalanine
Bip is biphenylalanine
Orn is ornithine
Dap is 2,3-diaminopropionic acid
Dab is 2,4-diaminobutyric acid
1-Nal is 1-naphthylalanine
2-Nal is 2-naphthylalanine
Ath is 2-amino-3-(anthracen-9-yl)propanoic acid
Phe(4,4′Bip) is 2-amino-3-[1,1′:4′,1″-terphenyl-4-yl]propionic acid

All of the molecules described herein may be in salt, ester or amide form.

Thus, also provided according to the present invention is a compound selected from the group consisting of: LTX-301, LTX-303 to LTX-310, LTX-312 to LTX-321, LTX-323-LTX-327, LTX-329, LTX-331-LTX-336, and LTX-338, or a salt, ester or amide thereof. Thus, the present invention provides a compound having a formula selected from the group consisting of: SEQ ID NOs: 10 and 12 to 42, or a salt, ester or amide thereof. Especially preferred is the compound LTX-315.

The molecules are preferably peptides and preferably have a modified, particularly an amidated, C-terminus. Amidated peptides may themselves be in salt form and acetate forms are preferred. Suitable physiologically acceptable salts are well known in the art and include salts of inorganic or organic acids, and include trifluoracetate as well as acetate and salts formed with HCl.

The molecules described herein are amphipathic in nature, their 2° structure, which may or may not tend towards the formation of an α-helix, provides an amphipathic molecule in physiological conditions.

### Molecules Having a Disubstituted β Amino Acid

As described in patent publication WO2011/051692, the peptide, peptidomimetic or amino acid derivative may have a net positive charge of at least +2 and incorporating a disubstituted β amino acid, each of the substituting groups in the β amino acid, which may be the same or different, comprises at least 7 non-hydrogen atoms, is lipophilic and has at least one cyclic group, one or more cyclic groups within a substituting group may be linked or fused to one or more cyclic groups within the other substituting group and where cyclic groups are fused in this way the combined total number of non-hydrogen atoms for the two substituting groups is at least 12. The 2 substituting groups on the β amino acid are preferably the same.

Lipophilicity can be measured by a molecule's distribution in a biphasic system, e.g. liquid-liquid such as 1-octanol/water. It is well known in the art that polar substituents such as hydroxy, carboxy, carbonyl, amino and ethers decrease the partition coefficient in a biphasic system such as 1-octanol/water as they reduce lipophilicity; the lipophilic substituting groups will therefore preferably contain no more than two, more preferably one or no such polar groups.

A β amino acid has the amino group attached to the β carbon atom; genetically coded amino acids are α amino acids in which the amino group is attached to the α carbon atom. This arrangement lengthens by one atom per β amino acid the backbone of a peptide incorporating one or more β amino acids. In this arrangement the α and/or the β carbon atom can be substituted. The α or β carbon atom may be disubstituted; where the α carbon atom is disubstituted a β^{2,2} amino acid results and where the β carbon atom is disubstituted a β^{3,3} amino acid is generated. One substituting group on each of the α or β carbon atoms results in a β^{2,3} amino acid. β^{2,2} and β^{3,3} disubstituted amino acids are preferred, β^{2,2} disubstituted amino acids being especially preferred.

The β amino acid is substituted by two groups incorporating at least 7 non-hydrogen atoms. Preferably one, more preferably both of the substituting groups contains at least 8, more preferably at least 10 non-hydrogen atoms. These groups are lipophilic in nature and while they may be different, are preferably the same. Each contains at least one cyclic group, typically a 6 membered ring which may be aliphatic or aromatic, preferably aromatic, and may be substituted, substituting groups may include hetero atoms such as oxygen, nitrogen, sulphur or a halogen, in particular fluorine or chlorine. Preferred substituting groups include C₁-C₄ alkyl (especially t-butyl), methoxy, fluoro and fluoromethyl groups. The cyclic groups may be homo-or heterocyclic, preferably they are homocyclic ring of carbon atoms. Preferred lipophilic substituting groups incorporate two or three cyclic groups, preferably two cyclic groups, which may be connected or fused, preferably fused. Particularly preferred substituting groups comprise a naphthalene group.

A further preferred group of lipophilic substituting groups have a single substituted or unsubstituted cyclic group, preferably a phenyl or cyclohexyl group.

The cyclic group or groups is typically spaced away from the peptide backbone (i.e. from the α or β carbon atom of the β amino acid) by a chain of 1 to 4, preferably 1 to 3 atoms; these linking atoms may include nitrogen and/or oxygen but will typically be carbon atoms, preferably the linking atoms are unsubstituted. These spacers are of course part of the substituting groups as defined herein.

Each substituting moiety of the disubstituted β amino acid will typically comprise 7 to 20 non-hydrogen atoms, preferably 7 to 13, more preferably 8 to 12, most preferably 9-11 non-hydrogen atoms.

These molecules will preferably be peptides or peptidomimetics of 1 or 2 to 12 amino acids or equivalent subunits in length. Unless otherwise clear from the context, reference herein to ‘amino acids’ includes the equivalent subunit in a peptidomimetic. The preferred molecules have either 1 to 3 or 4 amino acids, but alternatively may be 3 to 12, preferably 5 to 12 amino acids in length. Molecules of use according to the invention may only comprise a single amino acid but this will be a ‘modified’ amino acid in order to fulfil the requirements for charge.

Single amino acids as well as peptides and peptidomimetics will preferably incorporate a modified C terminus, the C terminal modifying group typically resulting in charge reversal, i.e. removing the negative charge of the carboxyl group and adding a positive charge, e.g. through the presence of an amino group. This modification alone, assuming the N terminus is not modified, will give the molecule overall a net charge of +2. Whether the C terminus is modified to give charge reversal or simply to remove the negative charge of the carboxyl group, the molecule preferably also contains one or more cationic amino acids. Thus the overall charge of the molecule may be +3, +4 or higher for larger molecules.

Suitable C-terminal groups, which are preferably cationic in nature, will typically have a maximum size of 15 non-hydrogen atoms. The C-terminus is preferably amidated and the amide group may be further substituted to form an N-alkyl or N, N-dialkyl amide. Primary and secondary amide groups are preferred. Suitable groups to substitute the amide group include aminoalkyl, e.g. amino ethyl or dimethylaminoethyl; the nitrogen atom of the amide group may form part of a cyclic group e.g. pyrazolidine, piperidine, imidazolidine and piperazine, piperazine being preferred, these cyclic groups may themselves be substituted, for example by alkyl or aminoalkyl groups.

Peptides preferably incorporate one or more cationic amino acids, lysine, arginine, ornithine and histidine are preferred but any non-genetically coded or modified amino acid carrying a positive charge at pH 7.0 may be incorporated.

Suitable non-genetically coded cationic amino acids and modified cationic amino acids include analogues of lysine, arginine and histidine such as homolysine, ornithine, diaminobutyric acid, diaminopimelic acid, diaminopropionic acid and homoarginine as well as trimethylysine and trimethylornithine, 4-aminopiperidine-4-carboxylic acid, 4-amino-1-carbamimidoylpiperidine-4-carboxylic acid and 4-guanidinophenylalanine.

Dipeptides will typically incorporate one cationic amino acid and longer peptides will usually incorporate additional cationic amino acids, thus a peptide of 4 or 5 amino acids may have 2 or 3 cationic amino acids and peptides of 6 to 9 amino acids may have 3 to 6 cationic amino acids.

A preferred group of molecules comprise a β^{2,2} disubstituted amino acid coupled to a C-terminal L-arginine amide residue and dipeptides having this arrangement are particularly preferred.

Peptides with three or more amino acids will typically have one or more additional lipophilic amino acids, i.e. amino acids with a lipophilic R group. Typically the lipophilic R group has at least one, preferably two cyclic groups, which may be fused or connected. The lipophilic R group may contain hetero atoms such as O, N or S but typically there is no more than one heteroatom, preferably it is nitrogen. This R group will preferably have no more than 2 polar groups, more preferably none or one, most preferably none.

Tryptophan is a preferred lipophilic amino acid and peptides preferably comprise 1 to 3 tryptophan residues. Further genetically coded lipophilic amino acids which may be incorporated are phenylalanine and tyrosine.

The lipophilic amino acids may be non-genetically coded, including genetically coded amino acids with modified R groups.

Especially preferred peptides, peptidomimetics or (modified) amino acids have a net positive charge of at least +2 and incorporate a group of formula I:

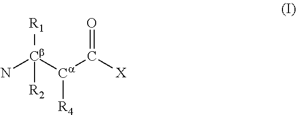

wherein any 2 from R₁, R₂, R₃ and R₄ are hydrogen atoms and 2 are substituting groups, which may be the same or different, comprise at least 7 non-hydrogen atoms, are lipophilic and include a cyclic group, said cyclic group not being attached directly either to the α or β carbon atom but optionally being linked or fused to a cyclic group in the other substituting group, where cyclic groups are fused the combined total number of non-hydrogen atoms for the two substituting groups is at least 12, and wherein X represents O, C, N or S.

It will be appreciated that the minimum figure of 12 for the combined total of non-hydrogen atoms in the two groups of R₁₋₄ when the cyclic groups of each moiety are fused is arrived at by adding the minimum number for the unfused groups (7+7=14) and subtracting 2 because two of the non-hydrogen atoms effectively participate in ring formation in each group. Preferably the combined total of non-hydrogen atoms in the two groups of R₁₋₄ when the cyclic groups of each moiety are fused is 14. Complex fused and linked groups can be envisaged where the two groups attached to the C^{α} or C^{β} may contain more than one pair of fused cyclic groups, with or without additional linking bonds between the substituting groups. Nevertheless, the two substituting groups are preferably not fused or linked as molecules in which these groups have greatest flexibility of movement are preferred.

The nitrogen atom in the group of formula (I) is preferably not bound to any atom of groups R₁₋₄, except, of course, indirectly through C^{β} or C^{α}. Preferably the 5 atoms in the above backbone (N—C^{β}—C^{α}—C—X) are connected to each other only in a linear, not cyclic, fashion. It will be appreciated that X and N in formula (I) have their normal valencies and thus will typically be further substituted as they are bound to other parts of the compound, e.g. further amino acids or N- or C-terminal capping groups.

The substituting groups of R₁₋₄ are generally lipophilic in nature and preferably carry no charge and preferably have no more than two, more preferably no more than one polar group. One or both of the substituting groups of R₁₋₄ preferably contain at least 8, more preferably at least 9 or 10 non-hydrogen atoms, e.g. 7-13, 7-12, 8-12 or 9-11 non-hydrogen atoms. These two substituting groups are preferably the same, if only for ease of synthesis. Preferably the two substituting groups are R₁ and R₂ or R₃ and R₄, R₃ and R₄ being most preferred.

As stated above, the cyclic groups of R₁₋₄ are not attached directly to either the a or B carbon atom because they are spaced therefrom by a chain of 1 to 4, preferably 1 to 3 atoms; these linking atoms may include nitrogen and/or oxygen but will typically be carbon atoms, preferably the linking atoms are unsubstituted. X may be substituted or unsubstituted and is preferably a N atom and preferably substituted. When X is N it may form part of an amide bond with a further amino acid. Alternatively, the N atom may be substituted, for example by an aminoalkyl group, e.g. aminoethyl or aminopropyl or dimethylaminoethyl. In a further alternative the N atom may form part of a cyclic group such as piperazine, which may itself be substituted by alkyl or aminoalkyl groups.

The peptides or peptidomimetics incorporating a group of formula I will preferably have a modified C terminus, which is preferably amidated and is described above.

Previous passages defining preferred substituting groups of the β amino acid apply, mutatis mutandis, to the two substituting groups of R₁₋₄. The peptides and peptidomimetics incorporating a group of formula I are a preferred sub-set of the molecules described earlier in this application and so all previous passages defining preferred characteristics of the molecules, for example their length and the other amino acids they contain, apply also to these molecules defined by their incorporation of a group of formula I, and vice versa. Particularly preferred molecules are 1 to 7 or 8 (e.g. 1 to 5), more preferably 1, 2, 3 or 4 amino acids in length. Peptidomimetic molecules will include the same number of subunits but these subunits will typically be linked by amide bond mimics; preferred linkages are discussed above and include esters and aminomethyl and ketomethylene.

The peptides, peptidomimetics and amino acids of the invention may be in salt form, cyclic or esterified, as well as the preferred amidated derivatives discussed above.

A preferred class of molecules are β, preferably β^{2,2}-amino acid derivatives which have a single β^{2,2}-amino acid incorporating two lipophilic side chains as defined above, the di-substituted β-amino acid being flanked by two cationic groups. As described previously, the two substituting groups are preferably the same, include a 6 membered cyclic group and at least 8, preferably at least 10 non-hydrogen atoms. Preferably, the molecule is LTX-401.

### Peptidomimetics

As discussed above, the molecules defined above may be in the form of a peptidomimetic. A peptidomimetic is typically characterised by retaining the polarity, three dimensional size and functionality (bioactivity) of its peptide equivalent but wherein the peptide bonds have been replaced, often by more stable linkages. By ‘stable’ is meant more resistant to enzymatic degradation by hydrolytic enzymes. Generally, the bond which replaces the amide bond (amide bond surrogate) conserves many of the properties of the amide bond, e.g. conformation, steric bulk, electrostatic character, possibility for hydrogen bonding etc. Chapter 14 of “Drug Design and Development”, Krogsgaard, Larsen, Liljefors and Madsen (Eds) 1996, Horwood Acad. Pub provides a general discussion of techniques for the design and synthesis of peptidomimetics. Suitable amide bond surrogates include the following groups: N-alkylation (Schmidt, R. et al., Int. J. Peptide Protein Res., 1995, 46, 47), retro-inverse amide (Chorev, M and Goodman, M., Acc. Chem. Res, 1993, 26, 266), thioamide (Sherman D. B. and Spatola, A. F. J. Am. Chem. Soc., 1990, 112, 433), thioester, phosphonate, ketomethylene (Hoffman, R. V. and Kim, H. O. J. Org. Chem., 1995, 60, 5107), hydroxymethylene, fluorovinyl (Allmendinger, T. et al., Tetrahydron Lett., 1990, 31, 7297), vinyl, methyleneamino (Sasaki, Y and Abe, J. Chem. Pharm. Bull. 1997 45, 13), methylenethio (Spatola, A. F., Methods Neurosci, 1993, 13, 19), alkane (Lavielle, S. et. al., Int. J. Peptide Protein Res., 1993, 42, 270) and sulfonamido (Luisi, G. et al. Tetrahedron Lett. 1993, 34, 2391).

The peptidomimetic compounds may have a number sub-units which are approximately equivalent in size and function to the sub-units of an equivalent lytic peptide. The term ‘amino acid’ may thus conveniently be used herein to refer to the equivalent sub-units of a peptidomimetic compound. Moreover, peptidomimetics may have groups equivalent to the R groups of amino acids and discussion herein of suitable R groups and of N and C terminal modifying groups applies, mutatis mutandis, to peptidomimetic compounds.

As is discussed in “Drug Design and Development”, Krogsgaard et al., 1996, as well as replacement of amide bonds, peptidomimetics may involve the replacement of larger structural moieties with di- or tripeptidomimetic structures and in this case, mimetic moieties involving the peptide bond, such as azole-derived mimetics may be used as dipeptide replacements. Peptidomimetics and thus peptidomimetic backbones wherein just the amide bonds have been replaced as discussed above are, however, preferred.

Suitable peptidomimetics include reduced peptides where the amide bond has been reduced to a methylene amine by treatment with a reducing agent e.g. borane or a hydride reagent such as lithium aluminium-hydride. Such a reduction has the added advantage of increasing the overall cationicity of the molecule.

Other peptidomimetics include peptoids formed, for example, by the stepwise synthesis of amide-functionalised polyglycines. Some peptidomimetic backbones will be readily available from their peptide precursors, such as peptides which have been permethylated, suitable methods are described by Ostresh, J. M. et al. in Proc. Natl. Acad. Sci. USA (1994) 91, 11138-11142. Strongly basic conditions will favour N-methylation over O-methylation and result in methylation of some or all of the nitrogen atoms in the peptide bonds and the N-terminal nitrogen.

Preferred peptidomimetic backbones include polyesters, polyamines and derivatives thereof as well as substituted alkanes and alkenes. The peptidomimetics will preferably have N and C termini which may be modified as discussed herein.

Peptidomimetic equivalents of all peptides described as preferred are also preferred.

### Synthesis of Molecules

The lytic molecules described herein may be synthesised in any convenient way. Generally the reactive groups present (for example amino, thiol and/or carboxyl) will be protected during overall synthesis. The final step in the synthesis will thus be the deprotection of a protected derivative of the invention.

In building up the peptide, one can in principle start either at the C-terminal or the N-terminal although the C-terminal starting procedure is preferred.

Methods of peptide synthesis are well known in the art but for the present invention it may be particularly convenient to carry out the synthesis on a solid phase support, such supports being well known in the art.

A wide choice of protecting groups for amino acids are known and suitable amine protecting groups may include carbobenzyloxy (also designated Z) t-butoxycarbonyl (also designated Boc), 4-methoxy-2,3,6-trimethylbenzene sulphonyl (Mtr) and 9-fluorenylmethoxy-carbonyl (also designated Fmoc). It will be appreciated that when the peptide is built up from the C-terminal end, an amine-protecting group will be present on the a-amino group of each new residue added and will need to be removed selectively prior to the next coupling step.

Carboxyl protecting groups which may, for example be employed include readily cleaved ester groups such as benzyl (Bzl), p-nitrobenzyl (ONb), or t-butyl (OtBu) groups as well as the coupling groups on solid supports, for example the Rink amide linked to polystyrene.

Thiol protecting groups include p-methoxybenzyl (Mob), trityl (Trt) and acetamidomethyl (Acm).

Preferred peptides of the invention may conveniently be prepared using the t-butyloxycarbonyl (Boc) protecting group for the amine side chains of Lys, Orn, Dab and Dap as well as for protection of the indole nitrogen of the tryptophan residues. Fmoc can be used for protection of the alpha-amino groups. For peptides containing Arg, 2,2,4,6,7-pentamethyldihydrobenzofurane-5-sulfonyl can be used for protection of the guanidine side chain.

A wide range of procedures exists for removing amine- and carboxyl-protecting groups. These must, however, be consistent with the synthetic strategy employed. The side chain protecting groups must be stable to the conditions used to remove the temporary α-amino protecting group prior to the next coupling step.

Amine protecting groups such as Boc and carboxyl protecting groups such as tBu may be removed simultaneously by acid treatment, for example with trifluoroacetic acid. Thiol protecting groups such as Trt may be removed selectively using an oxidation agent such as iodine.

References and techniques for synthesising peptidomimetic compounds are provided above and known to the skilled man.

The subject will typically be a human patient but non-human animals, such as domestic or livestock animals may also be treated and laboratory or test animals may be treated.

Preferred cancer targets are lymphomas, leukaemias, neuroblastomas and glioblastomas (e.g. from the brain), carcinomas and adenocarcinomas (particularly from the breast, colon, kidney, liver, lung, ovary, pancreas, prostate and skin) and melanomas.

The administered molecule may be presented, for example, in a form suitable for oral, topical, nasal, parenteral, intravenal, intratumoural, rectal or regional (e.g. isolated limb perfusion) administration. Administration is typically by a parenteral route, preferably by injection subcutaneously, intramuscularly, intracapsularly, intraspinaly, intratumouraly or intravenously. Intratumoural administration is preferred.

The molecules defined herein may be presented in the conventional pharmacological forms of administration, such as tablets, coated tablets, nasal sprays, solutions, emulsions, liposomes, powders, capsules or sustained release forms. Conventional pharmaceutical excipients as well as the usual methods of production may be employed for the preparation of these forms. Organ specific carrier systems may also be used.

Injection solutions may, for example, be produced in the conventional manner, such as by the addition of preservation agents, such as p hydroxybenzoates, or stabilizers, such as EDTA. The solutions are then filled into injection vials or ampoules.

Preferred formulations are in saline. Such formulations being suitable for local administration, e.g. intratumoural, e.g. by injection or by perfusion/infusion.

Dosage units containing the active molecules preferably contain 0.1-10 mg, for example 1.5 mg of the antitumour molecule of the invention.

In employing such compositions systemically, the active molecule is present in an amount to achieve a serum level of the active molecule of at least about 5 μg/ml. In general, the serum level need not exceed 500 μg/ml. A preferred serum level is about 100 μg/ml. Such serum levels may be achieved by incorporating the bioactive molecule in a composition to be administered systemically at a dose of from 1 to about 10 mg/kg. In general, the molecule(s) need not be administered at a dose exceeding 100 mg/kg.

The molecules of the invention include salt forms. Appropriate pharmaceutically acceptable salts for peptides and similar molecules are well known to those skilled in the art.

The invention is further described in the following Examples and with reference to the figures in which:

FIG. 1 shows the T-cell clonality mechanism of action of the lytic compounds described above. 1) Administration of lytic compound to a cold tumour leads to the release of potent immune-stimulants and a broad repertoire of tumour-specific antigens; 2) activation of antigen presenting cells and engulfment of tumour specific antigens; 3) presentation of the broad repertoire of tumour-specific antigens takes place in a lymph node; 4) clonality of tumour-specific T cells is enhanced, and these T cells are distributed via the blood system; and 5) increased T-cell infiltration and clonality makes both the originally injected tumour, and other distal non-injected tumours hot.

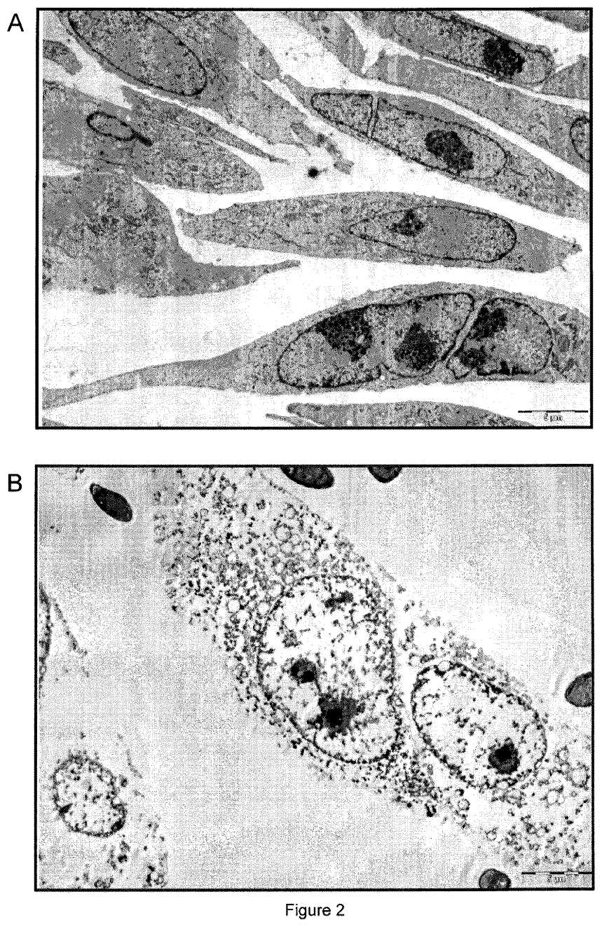

FIG. 2 shows the disintegration of the cytoplasmic membranes of osteoscarcoma cells after treatment with LTX-315. (A) shows the cells before lysis, (B) shows the cells after lysis.

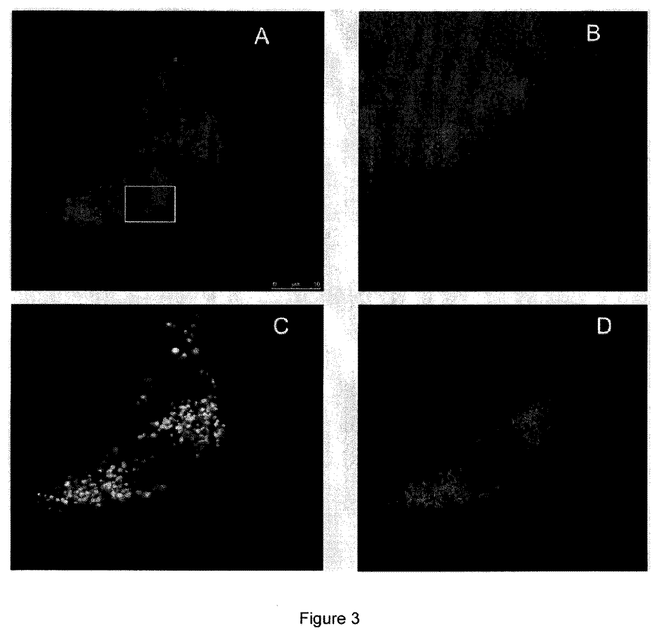

FIG. 3 shows internalization and accumulation of LTX-315 close to the mitochondria. A375 cells treated 30 minutes with 1.5 μM fluorescence-labelled LTX-315, and with labelled mitochondria and nucleus. The peptide was internalized and detected in close proximity to the mitochondria. A: overlay channels, B: close up, C: mitochondria. D: peptide

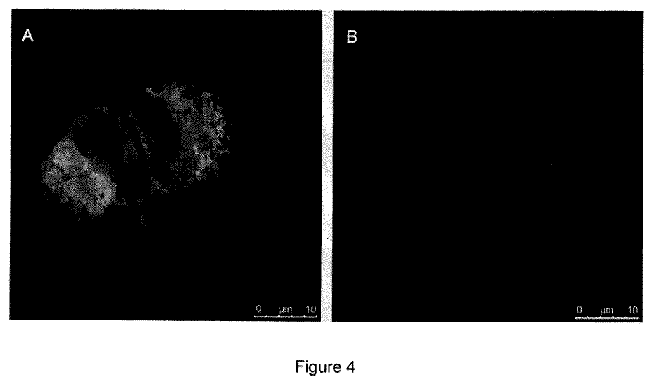

FIG. 4 shows that internalization occurs only in lytic 9-mer compounds such as LTX-315 and not in the non-lytic mock peptide LTX-328. A375 cells treated with 3 μM LTX-315 or LTX-328 peptide for 60 min. LTX-315 was detected in the cytoplasm, while LTX-328 was not internalized. A: LTX-315 60 min incubation, B: LTX-328 60 min incubation.

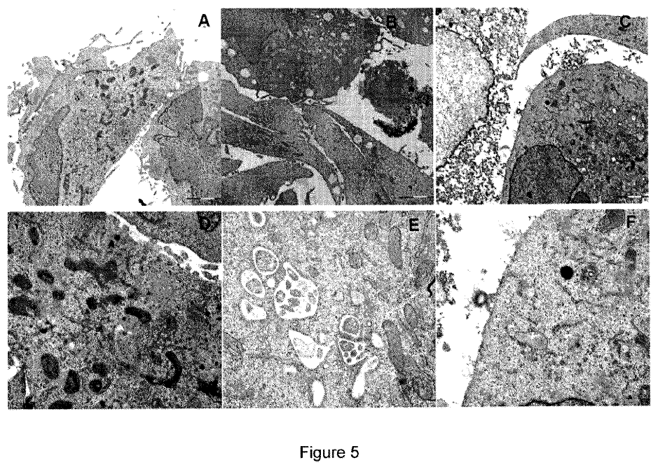

FIG. 5 shows that LTX-315 treatment causes ultrastructural changes. TEM images of A375 cells treated with LTX-315 for 60 minutes compared to control cells. A&D: untreated control cells, B&E: cells treated with 3,5 μM, C: &F cells treated with 17 μM. Magnification 10 000X A-C, 30 000 D-F, scale bar 5 μm.

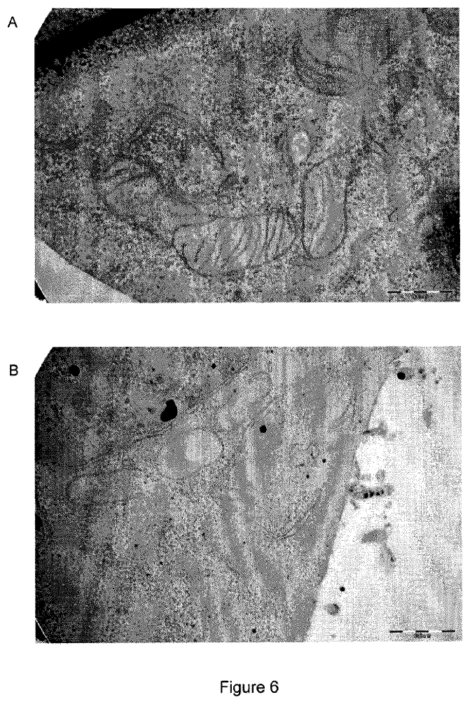

FIG. 6 shows that LTX-315 disintegrates the mitochondria membrane. TEM images of human A547 melanoma cells treated with LTX-315 (10 μg/ml) for 60 minutes compared to control cells.

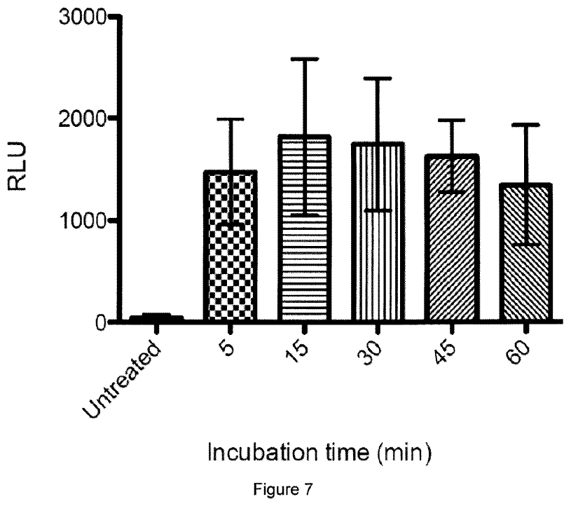

FIG. 7 shows the extracellular ATP levels following LTX-315 treatment: A375 cells were treated with LTX-315 for 5 minutes at different concentrations or maintained under controlled conditions, and the supernatant was analysed for the quantification of ATP secretion by luciferase bioluminescence. Quantitative data (mean+−S.D.) for one representative experiment are reported.

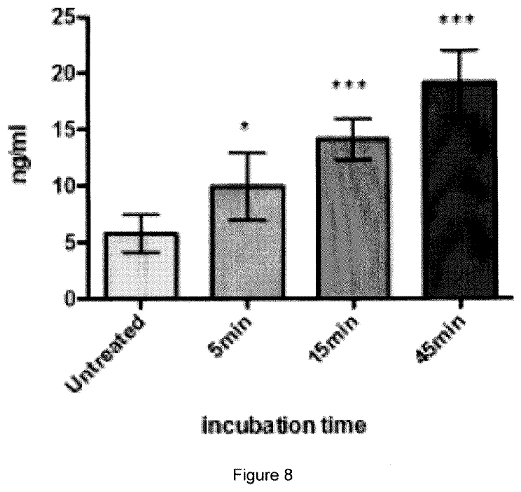

FIG. 8 shows that human melanoma cells treated with LTX-315 release cytochrome-C in the supernatant. Cytochrome-C release in the supernatant after LTX-315 treatment of A375 after designated time points (5, 15, 45 min) were determined by ELISA assay.

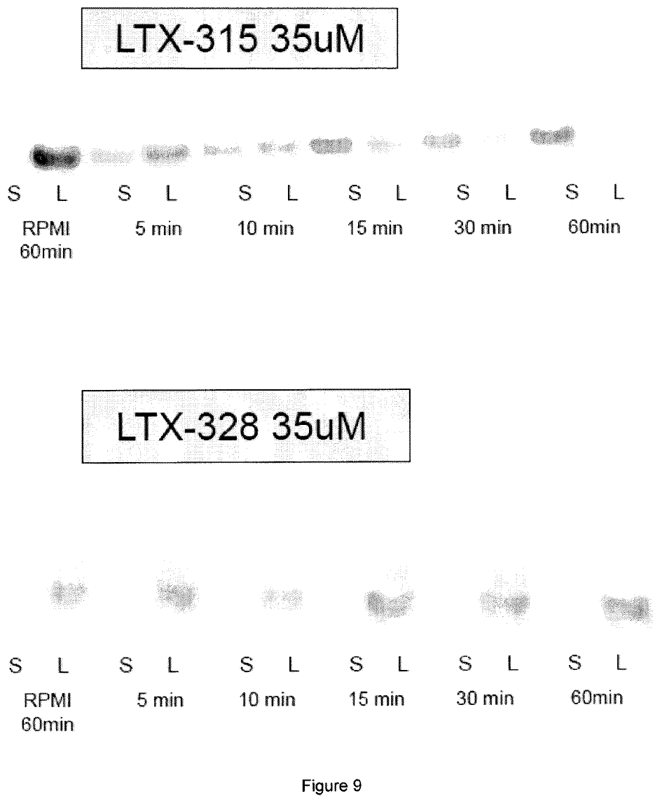

FIG. 9 shows that HMGB1 is released in the supernatant after LTX-315 treatment. A375 human melanoma cells were treated with 35 μM LTX-315 (top) or LTX-328 (bottom), and cell lysate (L) and supernatant(S) were analysed with Western blot, and the LTX-315-treated cells showed a gradual translocation from the cell lysate to the cell supernatant. Control cells were treated with media alone, and showed no translocation after 60 minutes.

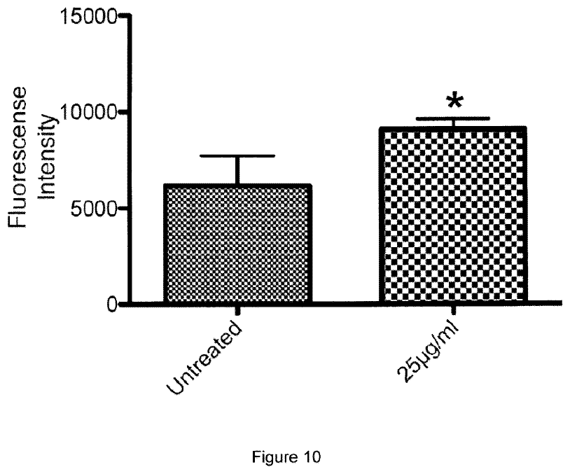

FIG. 10 shows that ROS generation in LTX-315 induced cell death. A375 cells were treated with LTX-315 at different concentrations for 15 minutes. After peptide treatment, carboxy-H2DCFDA was added to the samples and fluorescence was analysed with a fluorescence plate reader. The experiment was conducted in duplicate, with bars representing mean fluorescence+−S.D.

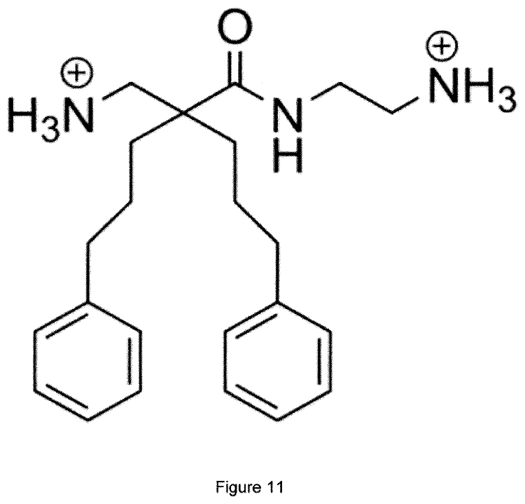

FIG. 11 shows the chemical structure of the small amphipathic β(2,2)-amino acid-derived antitumor molecule LTX-401 (MWₙₑₜ=367.53).

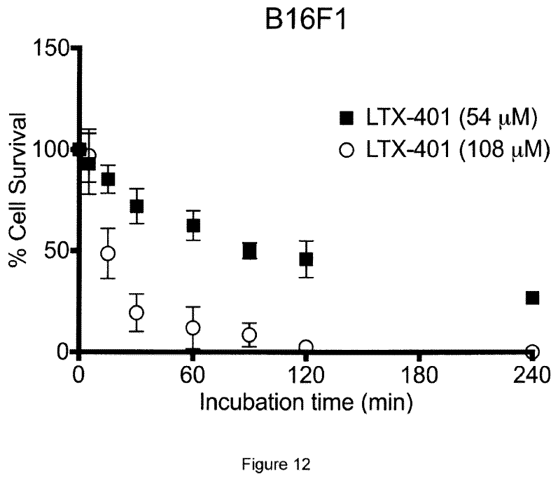

FIG. 12 shows that LTX-401 rapidly induces cell death in B16F1 melanoma cells. The determination of cell viability after a short incubation at two different concentrations of LTX-401 was analyzed after designated time points (5, 15, 30, 60, 90, 120 and 240 minutes), which revealed a decreased viability after 60 minutes of incubation. Data represent three experiments performed in triplicate presented for each time point as mean±SEM.

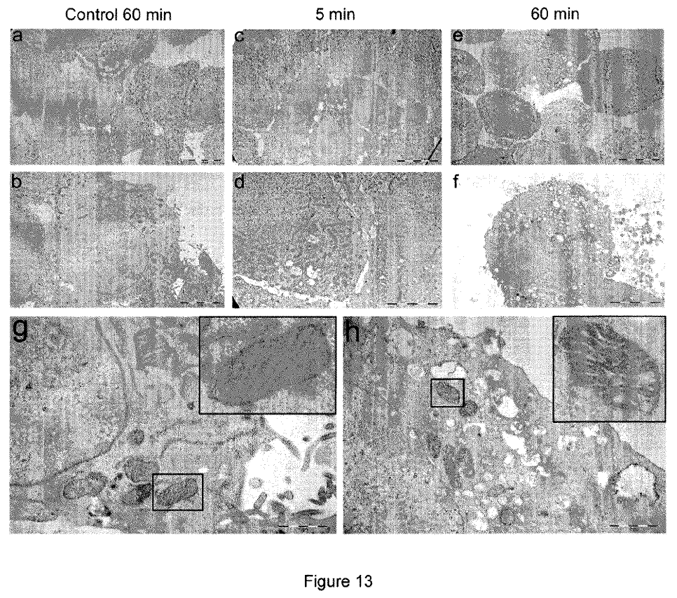

FIG. 13 shows that LTX-401 treatment induces ultrastructural changes with vacuolization. Representative TEM micrographs of B16F1 cells treated with LTX-401 (108 μM). Untreated control cells (a, b) were kept in a serum-free RPMI 1640 only until the experimental endpoint (60 minutes), and compared with cells treated for both 5 min (c, d) and 60 min (e, f); scale bars=10 μm for a, c, e, 5 μm for b, d, f.

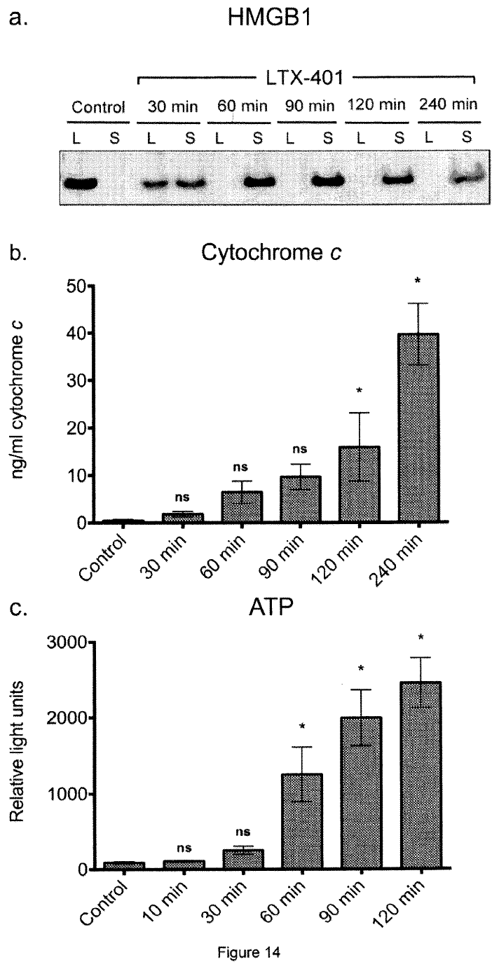

FIG. 14 shows that LTX-401 induces the release of danger signals. (a) Release of HMGB1 into the supernatant of LTX-401-treated cells as determined with Western blot. Translocation of the nuclear protein HMGB1 from the cell lysate (L) to the culture supernatant (S) was evident after 30 minutes of treatment with LTX-401 (108 μM), and the translocation was absolute after 90 minutes of incubation. Control cells showed no translocation after 240 minutes. Experiment was conducted thrice, and this figure is a digital image of a representative blot; (b) B16F1 cells were treated with LTX-401 (108 μM) for different time points (30, 60, 90, 120 and 240 minutes) before determining the amount of cytochrome c in supernatants using an ELISA assay. The results are presented as mean +/−SEM; (c) B16F1 cells were treated with LTX-401 (54 μM) for designated time points (10, 30, 60, 90 and 120 min). The quantification of ATP level was performed by luciferase bioluminescence, and the results are presented as mean +/−SEM.

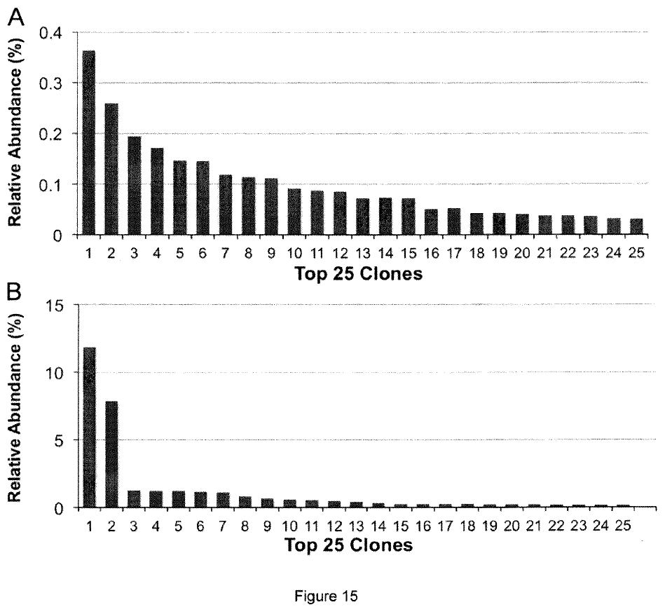

FIG. 15 shows examples of frequency distributions for T cell populations with varying levels of clonality. (A) shows a distribution curve of a population with a clonality of 0.05. (B) shows a distribution curve of a population with a clonality of 0.32.

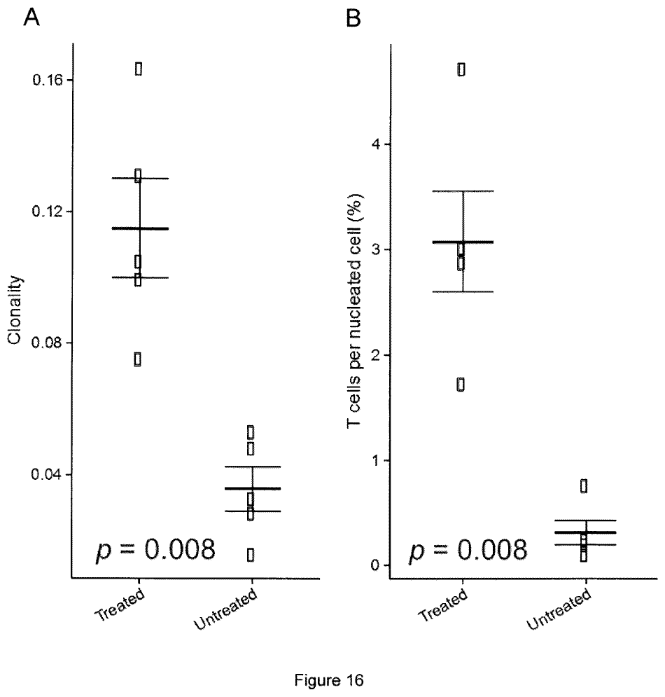

FIG. 16 shows the increase in clonality (A) and number of T cells per nucleated cell (B) in tumours following treatment with LTX-315. The p-value of 0.008 was calculated using a U test.

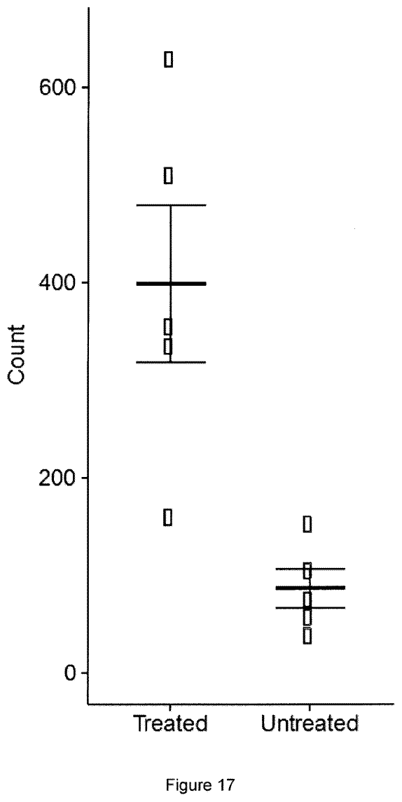

FIG. 17 shows the increase in T cell clone count in tumours following treatment with LTX-315. The p-value of 0.008 was calculated using a U test.

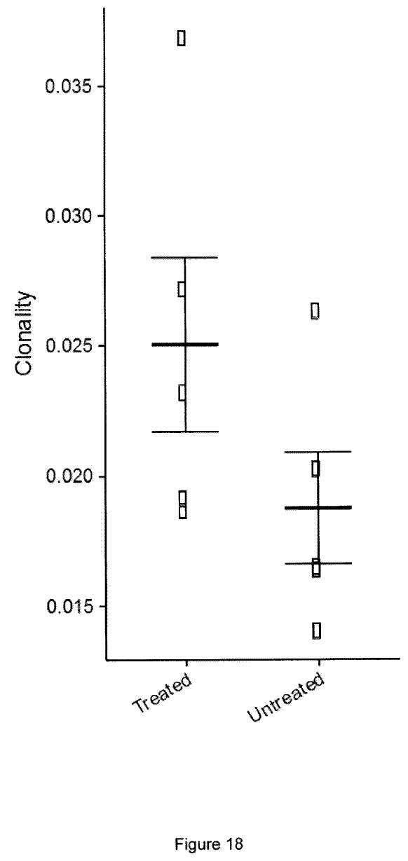

FIG. 18 shows a slight increase in clonality in peripheral blood mononuclear cells (PBMCs) following treatment with LTX-315. The p-value (0.15) is not significant if p<0.05.

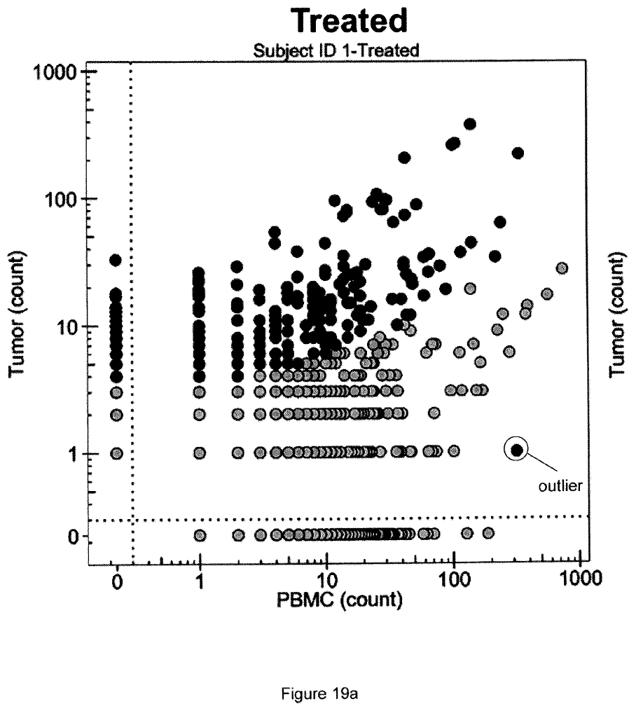

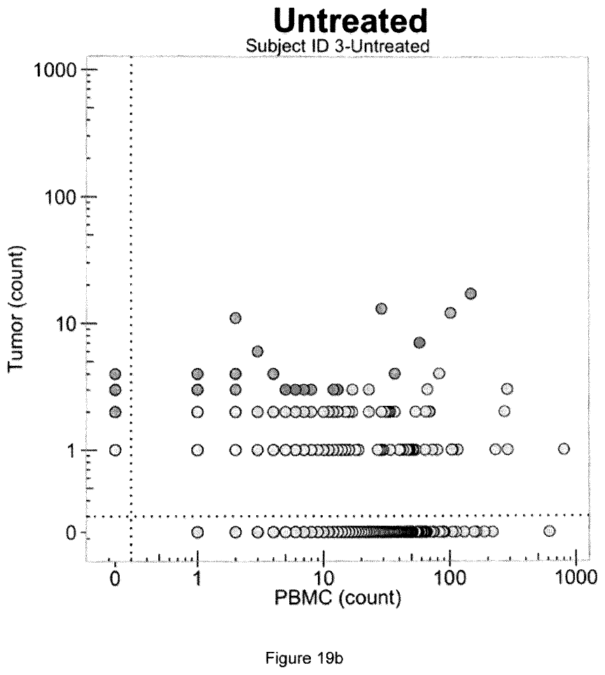

FIG. 19 shows repertoire comparisons between tumour (y-axis) and PBMCs (x-axis) from one example treated (FIG. 19a) and untreated (FIG. 19b) mouse subject. The black dots represent clones that have a significantly greater abundance in the tumour compared to in the PBMCs, apart from the one outlier labelled in FIG. 19a, which had a significantly greater abundance in the PBMCs compared to in the tumour. The grey dots represent clones showing no significant preference for the tumour or the PBMCs.

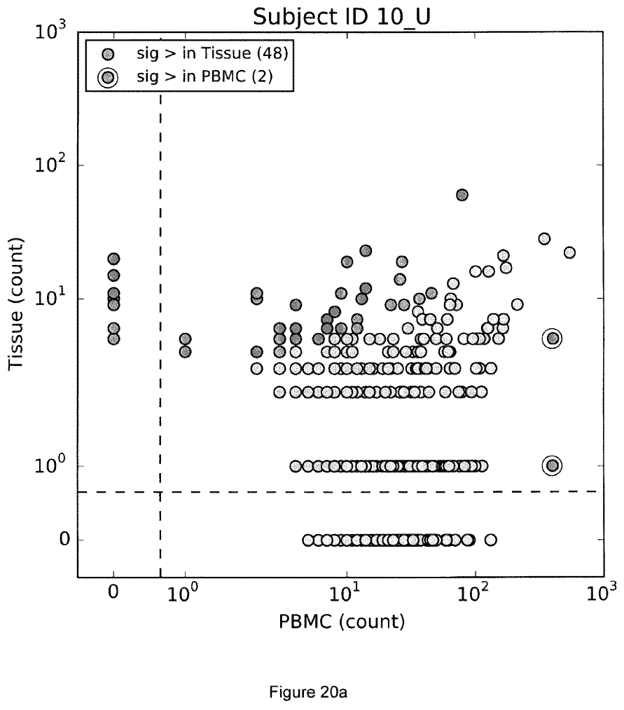

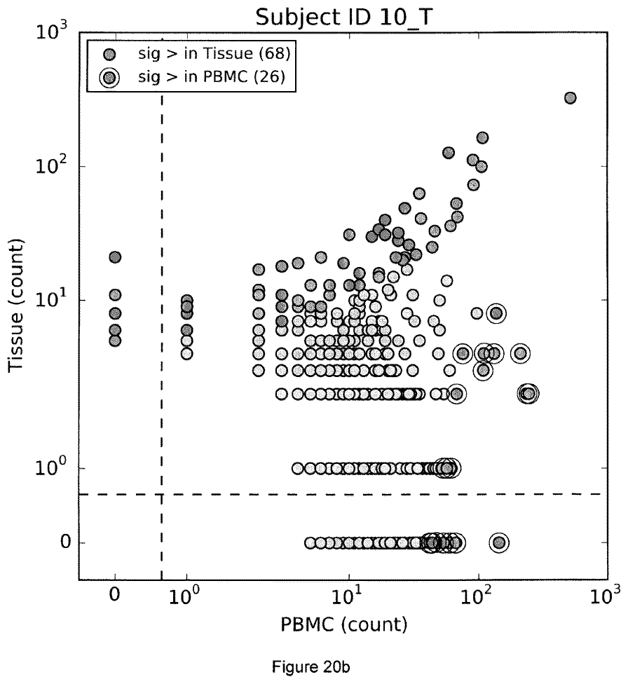

FIG. 20 shows repertoire comparisons between tumour tissue (y-axis) and PBMCs (x-axis) from one further example treated (FIG. 20b) and untreated (FIG. 20a) mouse subject. The dark grey dots represent clones that have a significantly greater abundance in the tumour compared to in the PBMCs, the light grey dots represents clones showing no significant preference for the tumour or the PBMCs and the dark grey dots with a halo represent clones that have a significantly greater abundance in the PBMCs compared to in the tumour.

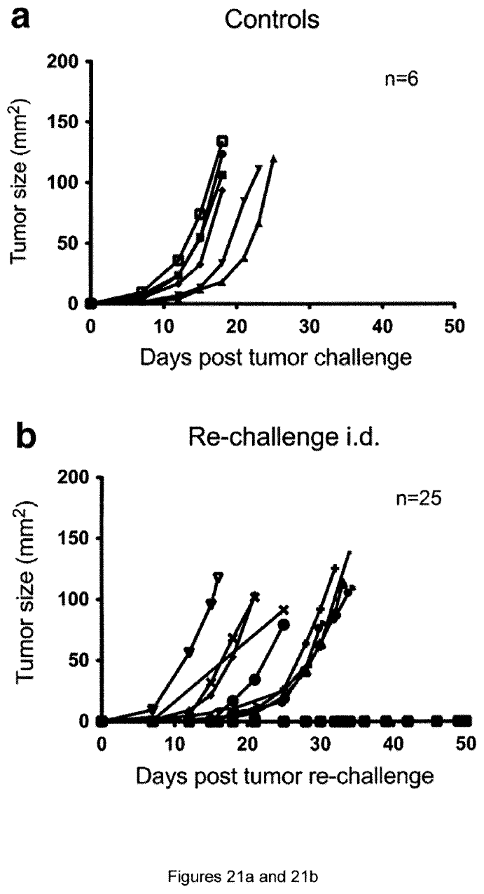

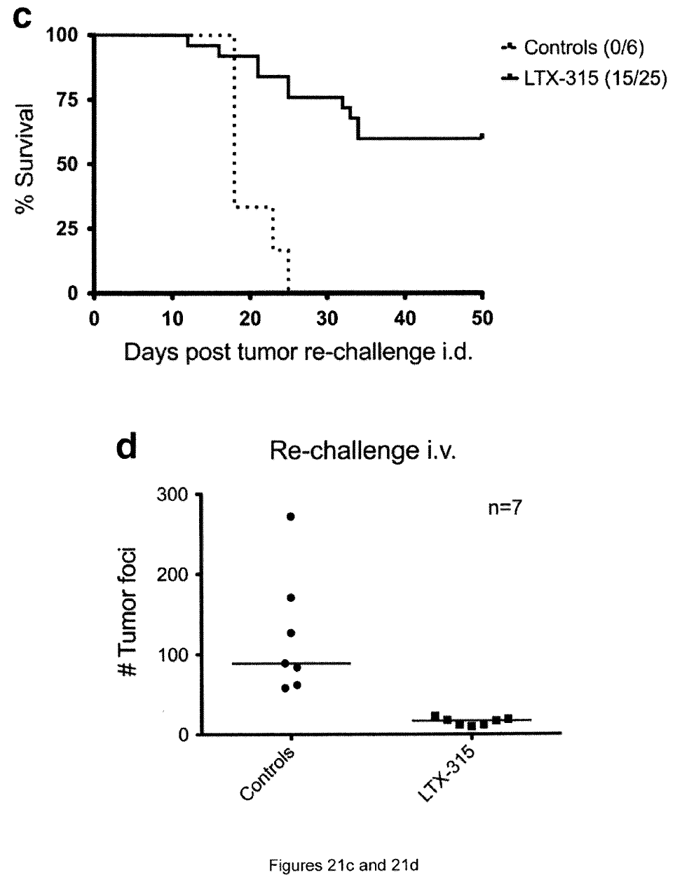

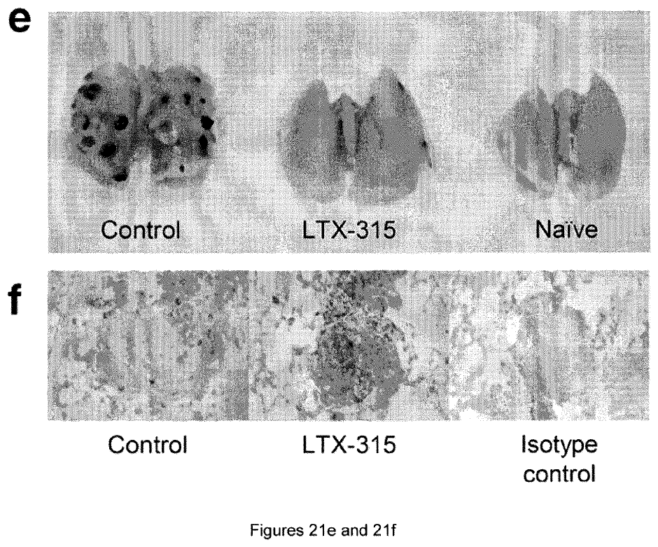

FIG. 21 shows that LTX-315 treatment induces immune protection against B16 melanomas. Tumour growth in non-treated control animals (a) was compared to animals previously cured by LTX-315 treatment (b) and (d). Animals were re-challenged intradermally with 5×10⁴ viable B16F1 cells contralateral to the first tumour site (b) or intravenously with 2×10⁵ viable B16F1 cells (d). The survival curves of animals re-challenged intradermally (c) were analysed using a log-rank (Mantel-Cox) test and were shown to be significantly different (p<0.0001). The number of lung tumour foci was analysed using an unpaired t test and was shown to be significantly different (p=0.003) when comparing intravenously re-challenged animals previously cured by LT X-315 with non-treated control animals (d). A digital image illustrates representative lungs from the different groups (e). The tumour foci of animals previously cured by LT X-315 were highly infiltrated by CD3+ T cells compared to control animals as shown by immunolabelling with anti-CD3 (f).

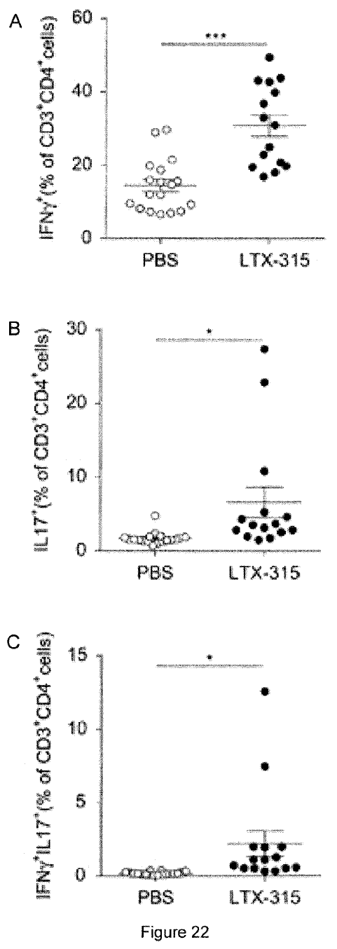

FIG. 22 shows that LTX-315 markedly increased the cytotoxic, CD4⁺ tumour infiltrating T lymphocytes (CD4⁺ TILs) in tumour beds. Flow cytometry determination of interferon γ positive (IFNγ⁺) CD4⁺ TILs (A), of interleukin 17 positive (IL-17⁺) CD4⁺ TILs (B) and of double-positive IFNγ⁺ IL-17⁺ CD4⁺ TILs (C) in the gate of live cells after dissociation of fresh MCA205 sarcoma 7 days post LTX-315 (versus PBS). Each dot represents data of one mouse; at least two experiments were gathered in each graph. Student's t-test: *=P<0.05 and ***=P<0.001.

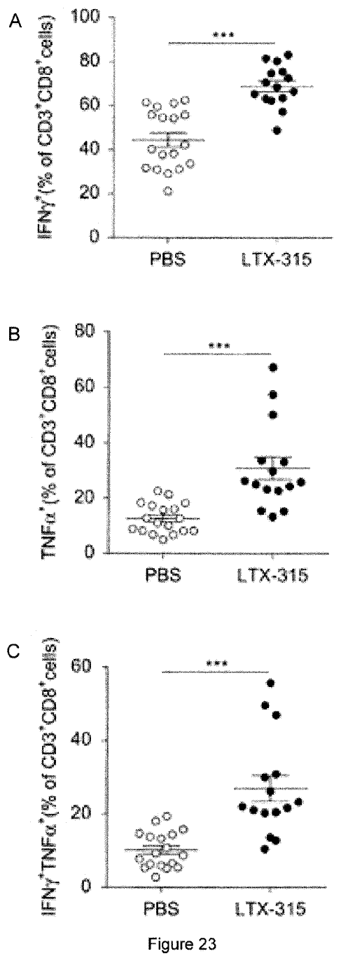

FIG. 23 shows that LTX-315 markedly increased the cytotoxic, CD8⁺ TILs in tumour beds. Flow cytometry determination of IFNγ⁺ CD8⁺ TILs (A), of tumour necrosis factor α positive (TNFα⁺) CD8⁺ TILs (B) and of double-positive IFNγ⁺ TNFα⁺ CD8⁺ TILs (C) in the gate of live cells after dissociation of fresh MCA205 sarcoma 7 days post LTX-315 (versus PBS). Each dot represents data of one mouse; at least two experiments were gathered in each graph. Student's t-test: *=P<0.05.

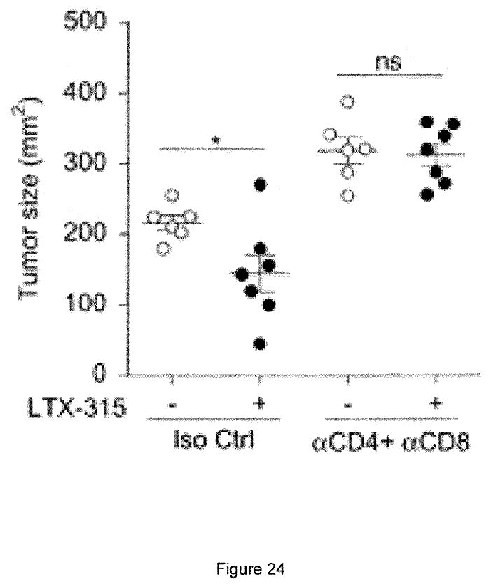

FIG. 24 shows the effects of depleting antibodies targeting CD4 and CD8a molecules on the tumouricidal activity of LTX-315 in 20-25 mm² established MCA205 sarcomas evolving in C57BL/6 immunocompetent mice. Tumour growth kinetics in the presence or absence of such specific antibodies (GK1.5 and 53-6.72, 200 μg per mouse) or isotype control mAbs injected. Three intratumoural daily consecutive injections of 300 μg of LTX-315 were then performed. A representative experiment out of two comprising 6 or 7 mice per group is shown. Student's t-test: *=P<0.05 and NS=not significant.

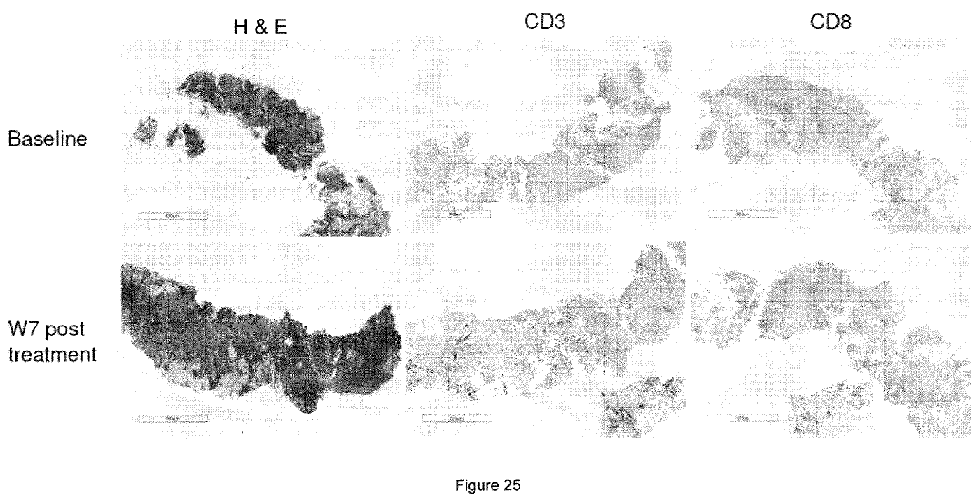

FIG. 25 shows hematoxylin & eosin (H&E) staining and infiltration of CD3⁺ positive cells (indicative of T lymphocytes) and CD8⁺ positive cells (indicative of cytotoxic T lymphocytes) after LTX-315 administration in a metastatic melanoma patient (as shown by the black dots).

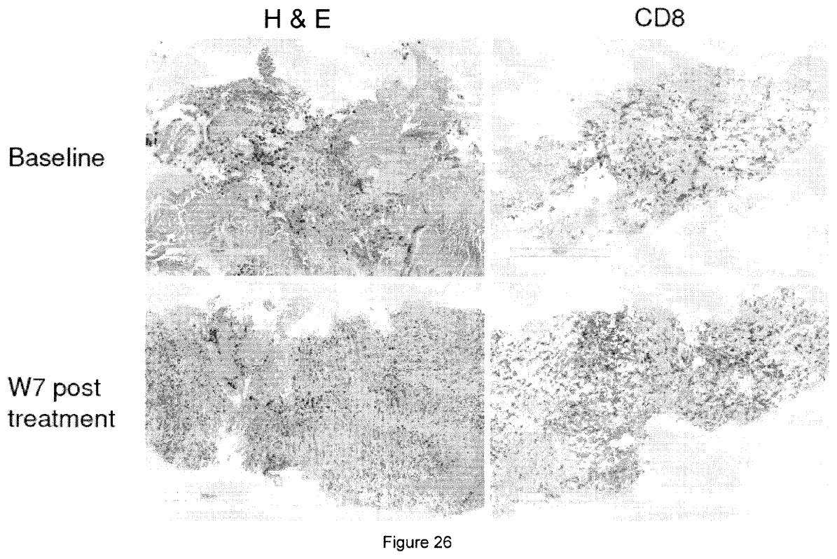

FIG. 26 shows hematoxylin & eosin (H&E) staining and infiltration of CD8⁺ positive cells (indicative of cytotoxic T lymphocytes) after LTX-315 administration in a malignant melanoma patient (as shown by the black dots).

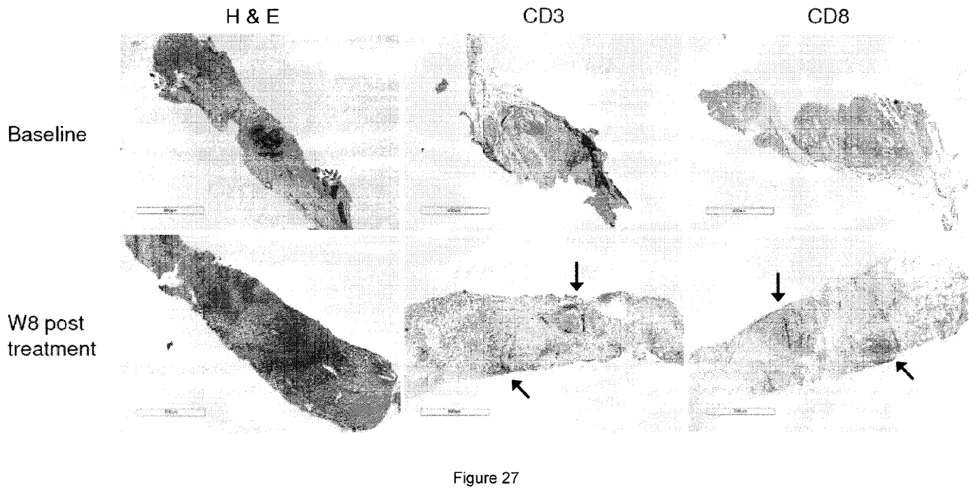

FIG. 27 shows hematoxylin & eosin (H&E) staining and infiltration of CD3⁺ positive cells (indicative of T lymphocytes) and CD8⁺ positive cells (indicative of cytotoxic T lymphocytes) after LTX-315 administration in a myo-epithelioma patient (as shown by the black arrows).

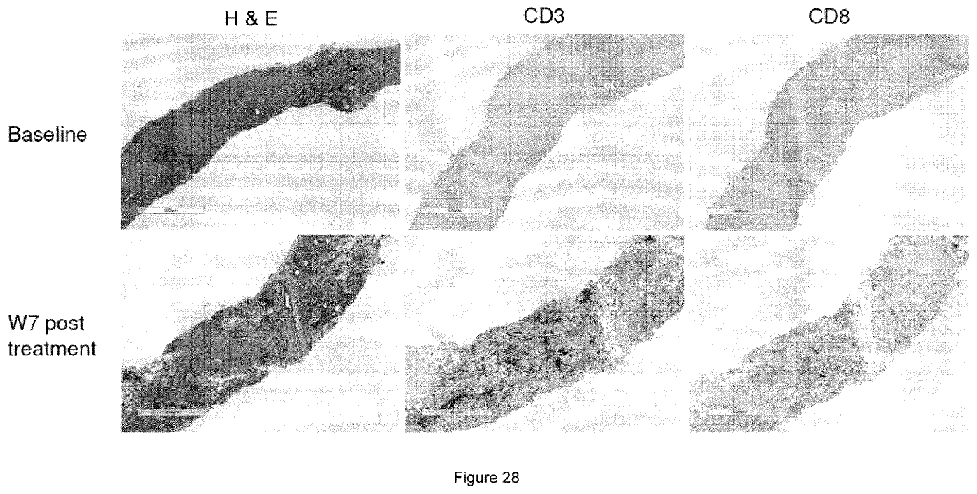

FIG. 28 shows hematoxylin & eosin (H&E) staining and infiltration of CD3⁺ positive cells (indicative of T lymphocytes) and CD8⁺ positive cells (indicative of cytotoxic T lymphocytes) after LTX-315 administration in a breast carcinoma patient (as shown by the black dots).

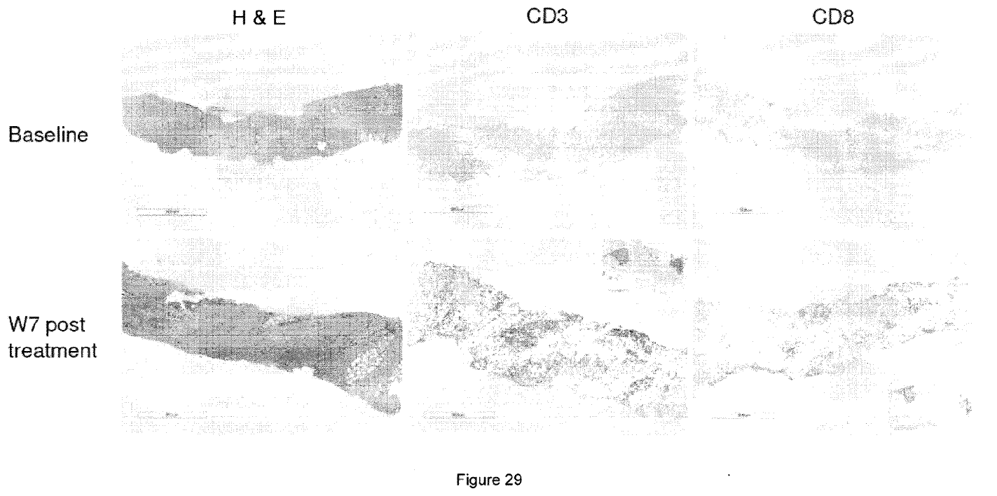

FIG. 29 shows hematoxylin & eosin (H&E) staining and infiltration of CD3⁺ positive cells (indicative of T lymphocytes) and CD8⁺ positive cells (indicative of cytotoxic T lymphocytes) after LTX-315 administration in a desmoid tumour patient (as shown by the black dots).

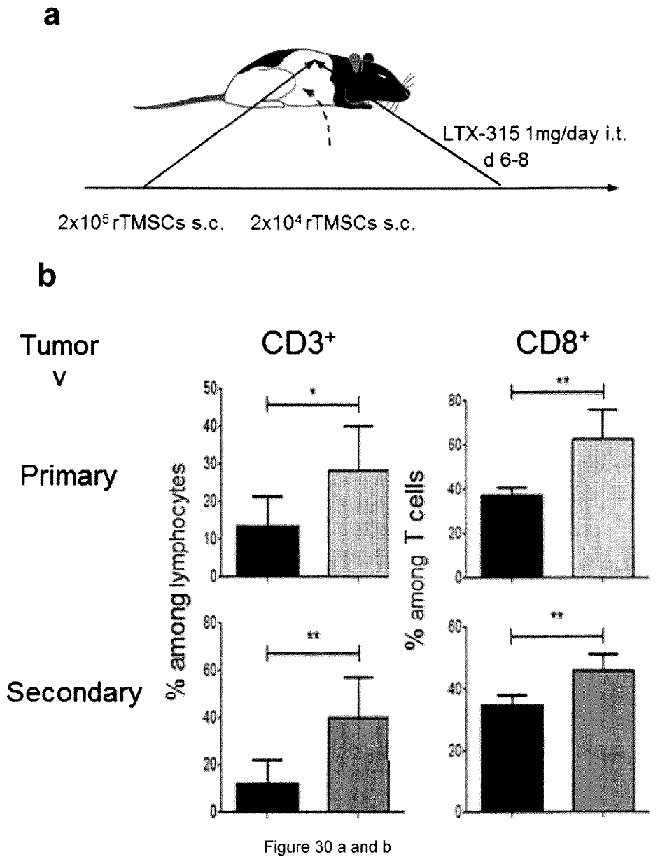

FIG. 30 shows that Infiltrating lymphocytes are recruited into the tumour after LTX-315 treatment. Schedule of experiment to study cell infiltration and modification of cell composition in tumours after LTX-315 treatment is shown in FIG. 30a. Rats were subcutaneously inoculated with 200,000 rat transformed mesenchymal stem cell-derived sarcoma model cells (rTMSCs) in one flank on day ÷2 and 20,000 rTMSCs in the opposite flank on day 0. Rats (n=8) with pre-established tumours were given intralesional injections of 1 mg LTX-315 daily for 3 subsequent days. Tumour-bearing rats in the control group (n=6) received saline injections in parallel. Single-cell suspensions were prepared from fresh tumour tissue at different time points during the week after LTX-315 treatment. FIG. 30b shows the percentage of TIL subsets in primary treated lesion (light grey bars), secondary lesion (dark grey bars) of treated rats compared with lesions from the control group (black bars). Graphs show mean±SD. * P<0.5, ** P<0.1 with Student's t test.

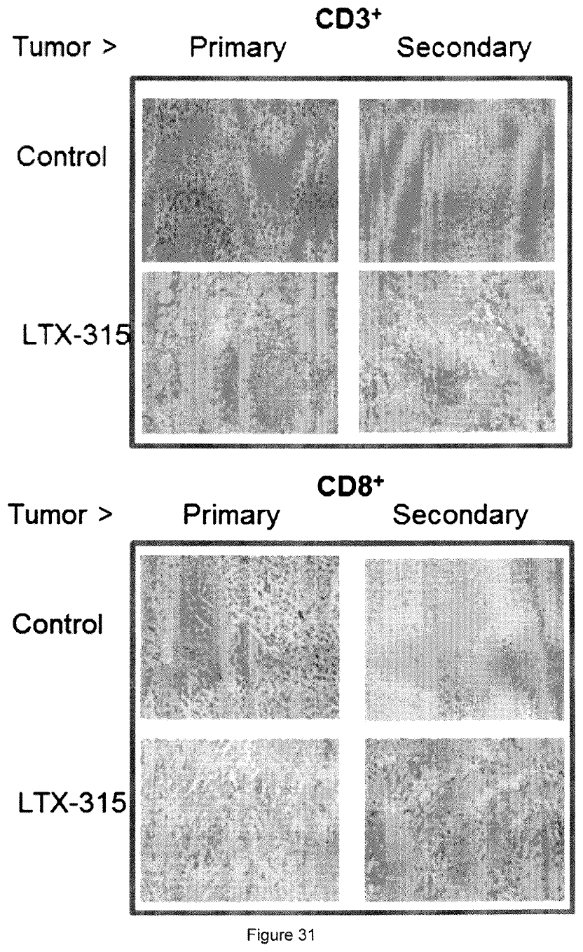

FIG. 31 shows representative images of primary and secondary tumours from treated rats versus untreated controls stained for CD3 or CD8 (brown) and counterstained with DAPI.

### Example 1

### LTX-315 Disintegrates the Cytoplasmic Membranes of Osteosarcoma Cells

### 1 Materials and Methods

Cytoplasmic membrane disintegration was carried out as described in FORVEILLE S. et al., Cell Cycle 2015, 14: 3506-12.

### 1.1 Chemicals and Cell Cultures

Media and supplements for cell culture were obtained from Gibco-Life Technologies (Carlsbad, CA, USA), chemicals from Sigma-Aldrich (St. Louis, MO, USA) with the exception of LTX-315 (K-K-W-W-K-K-W-Dip-K-NH2) that was provided by Lytix Biopharma (Tromsø, Norway); plastic ware from Greiner Bio-One (Monroe, CA, USA).

Human osteosarcoma U2OS cells were cultured in Glutamax-containing DMEM medium supplemented with 10% fetal calf serum (FCS), and 10 MM HEPES buffer. Cells were grown at 37° C. in a humidified incubator under a 5% CO₂ atmosphere.

### 1.2 Transmission Electron Microscopy

For ultrastructural studies, human osteosarcoma U2OS cells were fixed in 1.6% glutaraldehyde (v/v in 0.1 M phosphate buffer) for 1 h, collected by scraping, centrifuged and the pellet was post-fixed 1% osmium tetroxide (w/v in 0.1 M phosphate buffer). Following dehydration through a graded ethanol series, cells were embedded in Epon™ 812 and ultrathin sections were stained with standard uranyl acetate and lead citrate. Images were taken using a Tecnai 12 electron microscope (FEI, Eindhoven, the Netherlands).

### 2 Results

After administration of LTX-315, the majority of cells adopted a necrotic morphology with disintegration of cytoplasmic membranes (FIG. 2).

### Example 2

LTX-315 Internalises and Interacts with Mitochondria

In this study, we investigated the tumouricidal effect of LTX-315 on human melanoma cells. The peptide internalized and was shown in association with mitochondria, ultimately leading to a lytic cell death. The LTX-315 peptide treats solid tumours with intratumoural injections through a two-stage mode of action: the first is the collapse of the tumour itself, while the second is the released damage-associated molecular pattern molecules (DAMPs) from the dying tumour cell, which can induce a subsequent immune protection against recurrences and metastasis.

### 1 Materials and Methods

The study was carried out as described in EIKE L-V et al., Oncotarget 2015, 6: 34910-23.

### 1.1 Reagents

LTX-315 and LTX-328 (K-A-Q-Dip-Q-K-Q-A-W-NH₂) were made on request by Bachem AG (Bubendorf, Switzerland) and Innovagen (Lund, Sweden), respectively. LTX-315 Pacific Blue and LTX-328 Pacific Blue were purchased on request from Innovagen (Lund, Sweden) Norud (Tromsø, Norway), respectively.

### 1.2 Cell Culture

The A375 cell line A375 (ECACC, 88113005) is a human malignant melanoma derived from patient material, and was purchased from Public Health England (PHE Culture Collections, Porton Down, Salisbury, UK). Cells were maintained as monolayer cultures in high glucose 4.5% DMEM supplemented with 10% FBS and 1% L-glutamine, but not as antibiotics (complete media). The cell line was grown in a humidified 5% CO₂ atmosphere at 37° C., and was regularly tested for the presence of mycoplasma with MycoAlert (Lonza).

### 1.3 Confocal Microscopy

Live cell imaging with unlabeled cells—A375 cells were seeded at 10,000 cells/well in a complete media in Nunc Lab-Tec 8-wells chambered covered glass (Sigma) precoated with 25 μg/ml human fibronectin (Sigma) that were allowed to adhere overnight. Cells were washed twice with a serum-free RPMI, treated with peptide dissolved in RPMI and investigated using Bright on a Leica TCS SP5 confocal microscope, with a 63X/1.2W objective. The microscope was equipped with an incubation chamber with CO₂ and temperature control.

Fixed cells, mitotracker—Cells were seeded as for live cell imaging, and treated with Mitotracker CMH2XROS (Invitrogen) at 100 nm for 15 minutes prior to peptide treatment. Cells were treated with 17 μM LTX-315, with negative control serum-free RPMI only. After 60 min of incubation, cells were analyzed using a Zeiss microscope.

All confocal imaging experiments were subsequently conducted at least twice with similar results.

Fixed cells, fluorescence-labeled peptide—Subconfluential A375 cells were seeded at 8,000 cells/well as above, and transfected on the second day using the Lipofectamine LTX with Plus transfection reagents (Invitrogen) following the manufacturer's protocol. The mitochondria were labeled using the pDsRed2-Mito, and the nucleus was labeled using the GFP-Histon2B plasmid (Imaging Platform, University of Tromsø). A day after transfection, cells were washed twice with serum-free RPMI, and treated at different concentration and incubation periods with LTX-315 Pacific Blue or LTX-328 Pacific Blue. LTX-315 PB exhibited a similar cytotoxic profile as the unlabeled LTX-315 as determined by MTT assay. Control cells were treated with unlabeled LTX-315 and also with serum-free RPMI only. After incubation, cells were fixed with 4% paraformaldehyde in PBS, and the wells were covered with Prolong Gold antifade (Invitrogen). Cells were further analyzed by use of a Leica TCS SP5 confocal microscope, with a 693, 1.2 W objective. Pacific Blue, GFP and Ds Red were excited using UV, with 488 and 561 lasers, and fluorescence channels were sequentially detected using the following band passes: UV: 420-480 nm (with attenuation), 488: 501-550 nm and 561: 576-676 nm.

### 1.4 TEM Electron Microscopy

A375 cells were seeded at 1×10⁵ cells per well in 6-well plates and allowed to grow for three days to optimize membrane structures in the culture, and the media was changed on the second day. Cells were washed twice in serum-free RPMI before being treated with LTX-315 dissolved in serum-free RPMI at 5, 10 and 25 μg/ml, with serum-free RPMI as a negative control. Cells were then washed with PBS twice before fixation for 24 hours in 4° C. with 4% formaldehyde and 1% gluteralaldehyde in a Hepes buffer at pH 7.8. Dehydration and post-fixation protocols included incubation in a 5% buffered tannic acid and incubation in a 1% osmium-reduced ferrocyanide. Ultrathin sections were prepared, and uranyl acetate (5%) and Reynolds's lead citrate were used for staining and contrasting. Samples were examined on a JEOL JEM-1010 transmission electron microscope, and images were taken with an Olympus Morada side-mounted TEM CCD camera (Olympus soft imaging solutions, GmbH, Germany).

### 1.5 Fluorescence Measurement of Reactive Oxygen Species (ROS)

A DCFDA cellular reactive oxygen species detection assay kit was purchased from abcam®, and A375 cells seeded in a 96-well Costar black clear bottom plate with 20,000 cells per well incubated in 37° C. 16 hours prior to DCFDA assay. Cells were washed with a 100 μL/well of pre-warmed PBS one time, and incubated with 20 UM of DCFDA in a buffer solution supplied with the kit at 37° C. in a cell culture incubator for 45 min, and then washed again with a buffer solution of 100 μL/well. The cells were then stimulated with a 100 μL/well LTX-315 peptide dissolved in a buffer solution at concentrations of 17 μM for 30 min, and cells not treated were used as a negative control. The fluorescence intensity was determined at an excitation wavelength of 485 nm and an emission wavelength of 530 nm on a FLUOstar Galaxy plate reader.

### 1.6 Release of High Mobility-Group Box-1 (HMGB1)

A375 cells were seeded with 3×10⁵ cells/well in 6-well plates in a complete media, and allowed to adhere overnight. Cells were treated with LTX-315 or LTX-328 at 35 μM, and incubated at 37° C. and 5% CO₂ for different time points (5, 10, 15, 30, 60 min), and negative controls were serum-free RPMI-1650. Supernatants (S) were collected and centrifuged at 1,400 g for five minutes, and cell lysates (L) were harvested after washing with PBS twice and then subsequently lysed using a 4×Sample buffer (Invitrogen, number), 0.1 M DTT (Sigma number) and water. Supernatants were concentrated using Amicon Ultra 50 K centrifugal filters (Millipore UFC505024), and the cell lysate was sonicated. Both supernatants and lysate were boiled and resolved in a 10% sodium dodecyl sulfate polyacrylamide gel electrophoresis (SDS-PAGE), and then electro transferred to a polyvindiline difluoride (PVDF) membrane (Millipore). The membrane was blocked in 5% milk and incubated with the HMGB1 antibody (rabbit, polyclonal, abcam ab 18256); the membrane was then rinsed several times with TBST, incubated with a horseradish peroxidase (HRP)-conjugated secondary antibody (abcam ab6721), rinsed again with TBST and then developed using WB Luminol Reagent (Santa Cruz Biotechnology, Heidelberg, Germany).

### 1.7 Release of Cytochrome-C

A375 cells were seeded as with HMGB1 studies, and treated with 35 μM for different time points (5, 15, 45). Supernatants were collected and concentrated as with HMGB1 studies, and samples from the supernatants were analyzed using a 4.5 hour solid form Cytochrome C-Elisa kit (R&D Systems, USA, #DCTC0) following the manufacturer's description. Shortly thereafter, a 50% diluted sample was analyzed and the optical density was determined using a microplate reader set at 450 nm, and this reading was then subtracted from the reading at 540 nm. A standard curve was generated for each set of samples assayed. Samples were run in four parallels, and the cytochrome-c released into the supernatant was expressed as a fold over the level of cytochrome-c in the supernatant of untreated cells.

### 1.8 Release of ATP

The supernatant of LTX-315-treated A375 cells was analyzed using an Enliten ATP luciferase assay kit (Promega, USA). Cells were then seeded as with an ROS assay, and treated with LTX-315 in different incubation times, from 1 to 15 minutes with two parallels, which was then conducted three times. Negative controls were untreated A375 cells exposed to serum-free media alone. Samples were diluted at 1:50 and 1:100, and analyzed with a Luminoscan RT luminometer according to the manufacturer's protocol.

### 1.9 Statistical Analysis

All data represent at least two independent experiments with at least two parallels, which were expressed as the mean±SD. Cytochrome-C release and ATP release data was compared using one-way ANOVA and a multiple comparison test, and we considered the P-value <0.05 to indicate statistical significance.

### 2 Results

### 2.1 LTX-315 Internalizes and Targets the Mitochondria

To investigate the internalization and fate of the peptide within the cells, LTX-315 was labeled with Pacific Blue and incubated with cells at concentrations of 3 μM and 1.5 μM, respectively. The labeled LTX-315 rapidly penetrated the plasma membrane and at 1.5 μM, the peptide showed an accumulation around the mitochondria after 30 minutes of incubation but was not detected in the cell nucleus (FIG. 3). The labeled non-lytic mock-sequence peptide LTX-328 did not demonstrate any internalization at any concentration or incubation time tested (FIG. 4).

### 2.2 LTX-315 Induces Ultra-Structural Changes in Cells

We further evaluated the ultrastructural changes in treated cells by performing transmission electron microscopy (TEM), in which A375 cells were treated with either peptides dissolved directly in media or in media alone. A significant number of the cells treated with a low concentration (3.5 μM) of the LTX-315 peptide for 60 minutes showed vacuolization, as well as some altering of the mitochondrial morphology (FIG. 5). The mitochondria appeared to be less electron-dense, also exhibiting some degree of reorganization, with the cristae lying further apart or not visible at all. The number of necrotic cells in these samples was less than 5%. In these low concentrations, vacuolization of the cytoplasm was observed. Another common finding in these samples were peripherally placed vacuoles, which were lined with a single membrane layer containing a homogenous material (FIG. 5B). When cells were treated with higher concentration (17 μM) for 60 min, approximately 40% of them displayed a necrotic morphology with a loss of plasma membrane integrity (FIG. 5C&E). The cells that were still intact displayed a great heterogeneity, from a normal appearance with microvilli to a round appearance, with mitochondria clearly affected. In this high concentration, only 4% of the cells investigated displayed vacuolization, and chromatin condensation was not visible in this material at any peptide concentration tested. These results demonstrate that LTX-315 kills the tumor cells with a lytic mode of action, while lower concentrations cause the cells to undergo ultrastructure changes, such as vacuolization and an altered mitochondrial morphology. Moreover, no significant morphological changes suggestive of apoptotic cell death were observed.

In a separate experiment, exposure of LTX-315 at 10 μg/ml to human A547 cells (an ovarian melanoma cell line) led to disintegration of the mitochondrial membrane (FIG. 6).

### 2.3 LTX-315 Treatment Leads to Extracellular ATP Release

DAMPs are molecules that are released from intracellular sources during cellular damage. DAMPs can initiate and perpetuate an immune response through binding to Pattern Recognition Receptors (PRRs) on Antigen Presenting Cells (APCs). Among commonly known DAMPs are ATP, HMGB1, Calreticulin, Cytochrome C, mitochondrial DNA and Reactive oxygen species (ROS). We next wanted to investigate whether ATP was released into the supernatant from cells treated with LTX-315. Hence, the supernatant from treated and non-treated cells analyzed using luciferase detection assay. As shown in FIG. 7, ATP was detected in the supernatant as early as after 5 minutes of treatment with LTX-315, and the release was concentration-dependent.

### 2.4 LTX-315 Treatment Induces Cytochrome-C Release in Supernatant

To assess whether LTX-315-treated cells released cytochrome-C into the medium, A375 cells were treated with LTX-315 at 35 μM at different time points (5, 15, 45 min). The supernatant was subsequently analyzed using an ELISA assay. Cells treated with 35 μM value had three times more cytochrome-C in the supernatant compared to untreated control cells. The increase in cytochrome-C was detected after only five minutes of treatment, and there was also an increase after 15 and 45 minutes of peptide treatment, respectively (FIG. 8).

### 2.5 LTX-315 Treatment Leads to Extracellular HMGB1 Release

HMGB1 is a non-histone, chromatin-binding nuclear protein. Once passively released from necrotic cells, HMGB1 is able to trigger the functional maturation of dendritic cells, cytokine stimulation and chemotaxis among several immunopotentiating effects.

HMGB1 is normally found in the cell nucleus and would be expected in a cell lysate of healthy cells, though not in the culture media (supernatant). In order to assess the release of HMGB1 from LTX-315-treated cells, we measured the translocation and free HMGB1 from the nuclear compartment within the cell lysate into the cell supernatant.

Both cell lysate and the cell supernatant of LTX-315- and LTX-328-treated A375 melanoma cells were analyzed using a Western blot. Cells were treated with 35 μM of either LTX-315 or LTX-328, with a gradual translocation from the cell lysate to the supernatant detected in the LTX-315-treated melanoma cells, but not in the cells treated with the mock sequence peptide LTX-328 or a serum-free medium only (FIG. 9).

### 2.6 LTX-315 Treatment Causes the Production of Reactive Oxygen Species (ROS) in A375 Melanoma Cells

The ROS generation following LTX-315 treatment was measured by CH2DCFDA fluorometric assay. Significant amounts of ROS were generated after 15 minutes of incubation with LTX-315, and the ROS levels were concentration-dependent (FIG. 10).

### 3 Discussion

LTX-315 labelled with the fluorescent molecule Pacific Blue was internalized within minutes after incubation with A375 melanoma cells, and was distributed in the cytoplasm (FIG. 3). At low concentrations, accumulation of the peptide around the mitochondria was evident, whereas at higher concentrations the peptide was more spread within the cytoplasm and accumulated in circular structures closer to the cell membrane. If the peptide attacks the mitochondrial membrane, a decrease or even a total collapse of the mitochondrial membrane potential would be expected. A confocal imaging of cells with the membrane potential-dependent mitochondrial stain Mitotracker CMXh2ROS showed a loss of mitochondrial signal a short time after peptide treatment (data not shown). The loss of the signal shows that the peptide interaction with the mitochondria causes a loss of mitochondrial membrane potential, which is crucial for the mitochondria's most important cellular functions. An altered mitochondrial morphology was also demonstrated with TEM. Cells treated with LTX-315 for 60 minutes had less electron-dense mitochondria with an altered organization of the cristae, as well as vacuolization within the mitochondria compared to untreated cells (FIG. 6). Furthermore, vacuolization was evident in approximately 20% of cells treated with 3.5 μM of LTX-315. When the mitochondria are dysfunctional, free oxygen radicals (ROS) may be formed, and by using fluorometric assays we demonstrated ROS formation within a few minutes after peptide treatment (FIG. 10).

In this study, we demonstrate that treatment with the LTX-315 peptide causes an increase in ROS levels in A375 melanoma cells after treatment. One explanation for these higher levels of ROS following peptide treatment could be that the peptide enters the cells and targets the mitochondria, and the dysfunctional mitochondria then releases ROS. Through an ELISA assay, we detected the release of cytochrome-C in the supernatant of peptide-treated cells after only a few minutes of treatment (FIG. 8). Cytochrome-C is a mitochondrial protein released from the intermembrane space and into the cytosol when the outer mitochondrial membrane is perturbed, and by binding to the apoptotic protease activating factor-1(Apaf-1) it is also a part of the apoptotic cascade that eventually leads to cell death by apoptosis. However, if cytochrome-C is found in the extracellular space, it has been reported to act as a pro-inflammatory mediator, thus activating NF-kB and inducing cytokine and chemokine production. The transition of HMGB1 from the cellular compartment to the extracellular compartment was detected using a western blot (FIG. 9). When the nuclear protein HMBG1 is released into the extracellular fluid, it functions as a DAMP, and can bind to both the PRR TLRs and to the RAGE receptors; the activation of these may lead to a number of inflammatory responses such as the transcription of pro-inflammatory cytokines. We also detected ATP released in the supernatant after peptide incubation (FIG. 7), and presented extracellularly it functions as a DAMP by activating the purinerg P2RX7 receptors on the DC. This receptor not only functions as a pore that opens for small cationic and later bigger molecules after binding to ATP, its activation also causes the processing and release of the pro-inflammatory cytokine IL-1β.

In summary, our data suggests that LTX-315 induces lytic cell death in cancer cells, not only by direct attack on the plasma membrane, but also as a result of an injury to vital intracellular organelles after the internalization of the peptide at concentrations too low to cause an immediate loss of plasma membrane integrity. We demonstrate that the peptide treatment causes the release of several DAMPs such as CytC, ATP, HMGB1 and ROS. The DAMPs may affect the cellular integrity of the damaged cells in several ways, but are also associated with so-called immunogenic cell death. The release of tumor-specific antigens into the extracellular compartment, together with potent immune stimulatory molecules (DAMPs) such as ATP, CytC and HMGB1, can give a strong immune response. In turn, these factors will lead to a maturation and activation of DCs and other accessory cells of the adaptive immune system.

### Example 3

LTX-401 Induces DAMP Release in Melanoma Cells

In the present study we examined the ability of the amino acid derivative LTX-401 to induce cell death in cancer cell lines, as well as the capacity to induce regression in a murine melanoma model. Mode of action studies in vitro revealed lytic cell death and release of danger-associated molecular pattern molecules, preceded by massive cytoplasmic vacuolization and compromised lysosomes in treated cells. The use of a murine melanoma model demonstrated that the majority of animals treated with intratumoural injections of LTX-401 showed complete and long-lasting remission. Taken together, these results demonstrate the potential of LTX-401 as an immunotherapeutic agent for the treatment of solid tumours.

### 1 Materials and Methods

### 1.1 Reagents

LTX-401 (Mwₙₑₜ=367.53) was synthetized on request by Synthetica AS (Oslo, Norway). The chemical structure is shown in FIG. 11.

### 1.2 Cell Cultures

JM1, a rat hepatocellular carcinoma, HEPG2 and BEL7402, both human hepatocellular carcinomas, were kindly provided by Dr. Pål-Dag Line, Director, Department of Transplantation Medicine, Oslo University Hospital. B16F1 (ATCC, CRL-6323), a murine skin malignant melanoma, MDA-MB-435S (HTB-129), a human malignant melanoma originating from a breast metastases, Malme-3M (HTB-64), a human malignant melanoma derived from a lung metastases, MRC-5 (ATCC, CCL-171), normal human lung fibroblasts, SK-N-AS (ATCC, CRL-2137), a human neuroblastoma cell line derived from a bone marrow metastases, HT-29 (ATCC, HTB-38), a human colorectal adenocarcinoma and HUV-EC-C (ATCC, CRL-1730), a human vascular endothelium cell line, were all purchased from the American Type Culture Collection (ATCC-LGC Standards, Rockville, MD, USA). HaCat, a human keratinocyte cell line, was kindly provided by Dr. Ingvild Pettersen, Department of Host Microbe Interactions, University of Tromsø. A375, a malignant melanoma cell line of human origin, was purchased from Public Health England (PHE Culture Conditions, Porton, Down, Salisbury, UK). JM1, A375, BEL7402, HEPG2 and B16F1 were all maintained in culture media consisting of DMEM (high glucose), while SK-N-AS, HT-29 and MDA-MB-435S were cultured in an RPMI-1640 medium containing 2 mM L-glutamine and sodium bicarbonate. Malme-3M was cultured in RPMI-1640, containing 4 mM L-glutamine and 20% FBS (fetal bovine serum). Lastly, the three non-malignant cell lines, HUV-EC-C, MRC-5 and HaCat, were cultured, respectively, using the EGM-2 BulletKit (Lonza, MA), containing basal media and growth factors, MEM (normal glucose) also containing 2 mM L-glutamine and 1% non-essential amino acids and DMEM (normal glucose) containing 2 mM L-glutamine. All media were (if not specified otherwise) supplemented with 10% FBS, with the exception of the EGM-2 BulletKit, which is delivered with an aliquot of 10 ml FBS to yield a final concentration of 2% FBS in solution.

### 1.3 In Vitro Cytotoxicity, MTT Assay

The MTT assay was employed to investigate the in vitro cytotoxicity of LTX-401 against a selection of both cancer and non-malignant cell lines. Pre-cultured cells were seeded at a density between 1×10⁴-1.5×10⁴ cells/well, and the experiment was performed as previously described in Camilio K. A., et al., Cancer Immunol. Immunother, 2014, 63: 601-13. The results were calculated using the mean of three experiments, each with triplicate wells, and expressed as a 50% inhibitory concentration (IC₅₀).

### 1.4 Kill Kinetics

The killing kinetics of LTX-401 were studied against B16F1 melanoma cells, using both the 2×IC₅₀^{4h} and 4×IC₅₀^{4h} values corresponding to 54 μM and 108 μM, respectively. Cells were seeded as previously described for MTT assay, and incubated with LTX-401 solutions for 5, 15, 30, 60, 90, 120 and 240 minutes. Cells were washed once with 100 μl of serum-free RPMI-1640 after incubation, and further incubated in a 10% MTT solution (diluted in a serum-free RPMI-1640) for an additional 2 h.

### 1.5 TEM Electron Microscopy

B16F1 cells were seeded in 35 mm sterile tissue culture dishes at a density of 1×10⁴ cells in a volume of 2 ml of culture media, and left to adhere and grow in a cell incubator at 37° C., >95% humidity and 5% CO₂ conditions. When cultured cells reached an acceptable confluence (˜80-90%), they were incubated with the 4×IC₅₀^{4h} value of LTX-401 (108 μM) for different time points (5, 15, 30 and 60 minutes) and subsequently fixed in a PHEM buffered (0.1 M) solution containing 0.05% malachite green oxalate (Sigma), 0.5% glutaraldehyde (GA, Electron Microscopy Sciences) and 4% formaldehyde (FA, Electron Microscopy Sciences). Malachite green is more frequently being used as an additive to primary fixation in order to reduce lipid extraction commonly associated with sample processing. Samples were immediately loaded and processed en bloc using the PELCO Biowave Pro Laboratory Microwave (Ted Pella, Inc.), a newly introduced method considered to be advantageous over conventional bench protocols, since it reduces sample preparation from days to hours. For all microwave steps, samples were microwaved at 23° C. with a 50° C. cut-out temperature. Both malachite green/GA/FA and osmium-reduced ferrocyanide (0.8% K₃Fe (CN)₆/1% OsO₄) fixation steps were run in “power on/off” cycles of 2 minutes on, 2 minutes off (100 W), with vacuum.

Samples were rinsed four times with 0.1 M PHEM buffer between each step (two bench rinses and two final rinses for 40 seconds at 250 W) before being stained with 1% tannic acid (Electron Microscopy Sciences, PA, USA) and 1% aqueous uranyl acetate (Electron Microscopy Sciences, PA, USA) under “power on/off” cycles of 1 minute on, 1 minute off (150 W), with vacuum. Samples were rinsed as previously described between each staining procedure, in addition to being microwaved twice in water at 250 W (vacuum off). The dehydration of samples occurred through a graded ethanol series (25%, 50%, 70%, 90% and 100%), microwaving at 250 W for 40 seconds on each grade (vacuum off) before being immersed en bloc with a 50% Epon resin (AGAR, DDSA, MNA and DNP-30), and increased to 100% for the two final infiltration steps, 3 minutes each at 250 W (with vacuum cycle; 20 seconds on, 20 seconds off). Samples were then polymerized overnight at 60° C. Ultrathin sections (70 nm) were prepared and placed onto standard formvar, carbon-stabilized copper grids. Next, samples were examined on a JEOL JEM-1010 transmission electron microscope, and images were taken with an Olympus Morada side-mounted TEM CCD camera (Olympus soft imaging solutions, GmbH, Germany).

### 1.6 Release of High Mobility Group Box 1 (HMGB1)

B16F1 cells were seeded at a density of 2×10⁵ cells/well in 6-well plates in a complete medium, and allowed to adhere overnight. Cells were treated with 108 μM of LTX-401 (4×IC₅₀^{4h}), and incubated at 37° C. (>95% humidity and 5% CO₂) for different time points (10, 30, 60, 90 and 120 minutes). Serum-free RPMI 1640 was used as a negative control, and supernatants (S) were collected and centrifuged at 1,400 g for 5 min before being up-concentrated using Amicon Ultra-0.5 Centrifugal Filter units with Ultracel-50 membrane (Milipore, Norway). Cell lysates (L) were harvested after washing with 2 mL of serum-free RPMI-1640 and subjected to a lysis buffer (mastermix) containing 2×NuPAGE LDS Sample buffer (Invitrogen, Norway), a 1×NuPAGE Sample Reducing Agent (Invitrogen, Norway) and 50% sterile H₂0. Both supernatants and lysates were boiled at 70° C. for 10 min and resolved on NuPAGE Novex 4-12% Bis-Tris Gels (Millipore, Norway) before being electrotransferred to a polyvindiline difluoride (PVDF) membrane (Millipore, Norway). Membranes were blocked for 1 h using 5% non-fat dry milk in TBS-T and next hybridized with HMGB1 antibody (rabbit polyclonal to HMGB1—ChIP Grade, Abcam, UK) overnight at 4° C., followed by horseradish peroxidase-(HRP) conjugated secondary antibody (goat polyclonal anti-rabbit lgG, Abcam, UK) for an additional 2 h at room temperature before being developed using WB Luminol Reagent (Santa Cruz Biotechnology, Heidelberg, Germany), and imaged/scanned using ImageQuant LAS 4000 and ImageQuant software (GE Healthcare).

### 1.7 Release of Cytochrome c

B16F1 cells were plated onto 96-well culture plates as previously described above for the MTT assay. Cells were treated with 108 μM (4×IC₅₀^{4h}) for designated time points (30, 60, 90, 120 and 240 minutes), while control cells were preserved in serum-free RPMI-1640 only until the experimental endpoint. Supernatants were collected and diluted 1/5 in a serum-free RPMI 1640 before being evaluated using a Rat/Mouse Cytochrome c Quantikine ELISA kit (R&D Systems, USA), according to the instructions of the manufacturer. The concentration of cytochrome c was calculated by interpolation values on the provided standard curve.

### 1.8 Release of ATP

B16F1 cells were seeded as previously described for the MTT assay, and treated with the 2×IC₅₀^{4h} value of LTX-401 (54 μM) for different time points (10, 30, 60, 90 and 120 minutes). Serum-free RPMI 1640 treated cells functioned as a negative and blank control, respectively. Extracellular levels of ATP were measured at the end of the experiment using a luciferin-based ENLITEN ATP Assay kit (Promega, Madison, WI, USA), in which ATP-driven chemoluminescence was recorded on a Luminescence Microplate Reader (Labsystems Luminoskan®, Finland), and expressed as relative light units (RLU).

### 1.9 LysoTracker

Flow cytometry: B16F1 cells were seeded in 6-well plates and allowed to adhere overnight. Cells were then treated with the 1×IC₅₀^{4h} of LTX-401 (27 μM) for 60 minutes, with 40 nM LysoTracker DND-26 (Invitrogen) added in the last 5 minutes. Untreated cells were used as a control. Cells were trypsinized and investigated on a FACS Calibur Flow cytometer, and the results were processed using FlowJo Software (Tree Star, Inc., Ashland, OR, USA). PI was utilized in some experiments to gate away cells with compromised plasma membrane.

Confocal microscopy: B16F1 cells were seeded in 8-well plates (Nunc) at 1×10⁴ cells/well and allowed to adhere overnight. Next, cells were treated with 27 μM of LTX-401 for 60 minutes and labelled with LysoTracker DND-26 for 5 minutes and with Hoecsht33342 for nuclear staining. Cells were immediately investigated on a Zeiss Confocal microscope with a controlled temperature and atmosphere.

### 1.10 Statistical Analysis

Results are presented as a mean±standard error of mean (SEM) or standard deviation (SD) of at least two independent experiments. MTT assays were conducted twice with three parallels and cytochrome c assays were conducted twice with two parallels, while ATP assays were conducted three times with two parallels. Cytochrome c release-and ATP release data were compared using one-way ANOVA and a multiple comparison test, and we considered a P-value ≤0.05 to be considered statistically significant.

### 2 Results

2.1 LTX-401 rapidly Induces Cell Death

In this study, LTX-401 effectively reduced the viability of several tumor cell lines in vitro (data not shown). LTX-401 displayed the highest cytotoxic activity against the human malignant melanoma cell line MDA-MB-435S (13.5 μM), and was least active against the human hepatocellular carcinoma cell line HEPG2 (35.4 μM). For the remaining cell lines, LTX-401 exhibited similar IC₅₀ values, varying slightly within the range of 19-32 μM.

Kinetic experiments were performed to determine the time course of the cytotoxic activity of LTX-401 against B16 melanoma cells. Two different concentrations representing the 2×IC₅₀^{4h} and 4×IC₅₀^{4h} of LTX-401, i.e. 54 μM and 108 μM, respectively, were used to assess the dose-dependent effect. As revealed by initial pilot studies, these two concentrations were also employed for the majority of in vitro experiments, and in particular when studying the release of DAMPs, as lower concentrations failed to induce such a modality.

LTX-401 (108 μM) exhibited rapid killing kinetics, with a kill ratio of 50% after 15 min, followed by nearly 100% cell death two hours after start of treatment. In contrast, cells incubated with 54 μM of LTX-401 followed a gradual inhibition of cell viability, with a 50% cell survival after two hours (FIG. 12).

### 2.2 LTX-401 Treatment Causes Ultrastructural Changes in Melanoma Cells

To further investigate the mode of action underlying the cytotoxic activity of LTX-401, B16F1 cells were incubated with the 4×IC₅₀^{4h} of LTX-401 (108 μM) for 5 and 60 min, respectively. Untreated cells served as a control, and were incubated in a serum-free RPMI 1640 until the experimental endpoint (60 min). After incubation, all cells were fixed and prepared for TEM studies. TEM images of untreated B16F1 cells revealed a rough surface characterized by frequent microvillus-like protrusions on the plasma membrane (FIG. 13a, b). The cytoplasm consisted of several electron-dense mitochondria, and visibly smooth ER and Golgi apparatus (FIG. 13a, b). The majority of LTX-401 treated cells lost their surface morphology after 5 min, and the majority of cells were without significant alterations except for a slight vacuolization of the cytoplasm (FIG. 13c). After 60 minutes of incubation, treated cultures demonstrated a heterogeneous population of cells, including both heavily vacuolated and large non-vacuolated cells (FIG. 13e). Large portions of cells were necrotic, as was clearly seen by the loss of plasma membrane integrity and leakage of cell constituents (FIG. 13e, f). The chromatin structure was more or less unaffected by LTX-401 treatment, albeit slightly condensed in a number of cells. Furthermore, a higher magnification revealed no significant ultrastructural changes in the mitochondria of the majority of LTX-401 treated cells (FIG. 13h) compared to controls (FIG. 13g). Cells treated for 60 minutes (FIG. 13h) displayed intact inner and outer mitochondrial membranes, albeit with some degree of mitochondrial swelling. Overall, these observations demonstrate that LTX-401 kills by a lytic mode of action, accompanied by cellular swelling, mitochondrial swelling and vacuolization of the cytoplasm.

### 2.3 LTX-401 Treatment Results in the Release of DAMPs in Melanoma Cells

Next, we wanted to characterize the ability of LTX-401 to induce the release of DAMPs. HMGB1 is a non-histone nuclear protein, and when released extracellularly it can act as a DAMP by binding to toll-like receptors (TLRs) or receptor for advanced glycation end-products (RAGE). The release of HMGB1 from B16F1 cells into cell culture supernatants was assessed by Western blot analysis. The translocation of HMGB1 was detected, with the release of HMGB1 occurring after 30 min of treatment. Non-treated control cells displayed no release of HMGB1 into the supernatant, as seen by the complete detainment of the protein within the lysates (FIG. 14a).

Cytochrome c is considered a mitochondrial-derived DAMP, and extracellular cytochrome c has been reported to induce NF-kB activation and the release of proinflammatory cytokines. To study whether LTX-401 was able to induce the release of cytochrome c from LTX-401-treated B16F1 cells, an ELISA assay was employed to measure the amounts of cytochrome c in the cell culture medium after treatment. A quantitative analysis demonstrated the presence of cytochrome c in the supernatant following LTX-401 treatment with the 4×IC₅₀^{4h} value of LTX-401 (108 μM) (FIG. 14b) after 120 min of treatment. The release of cytochrome c followed a gradual increase over time, reaching a peak of approximately 40 ng/ml at the experimental endpoint (FIG. 14b).

ATP is reported to have immunogenic properties when released from dying and/or stressed cells, including cancer cells succumbing to conventional chemotherapy. To investigate whether LTX-401 was able to induce the release of ATP from B16F1 cells, a luciferin-luciferase-based reaction assay was employed. The extracellular concentration of ATP quickly rose 60 min after initiating treatment with a gradual increase towards 120 min (FIG. 14c).

2.4 Treatment with LTX-401-Induced Loss of Lysosomal Integrity in Melanoma Cells

Next, we wanted to investigate whether LTX-401 caused any changes in lysosomal integrity. Treatment with LTX-401 resulted in a loss of signal from the acidophilic dye LysoTracker DND-26, as shown with flow cytometry and confocal microscopy. This dye accumulates in acidic organelles such as lysosomes and melanosomes. The effect was shown not to be cell-type specific, as similar results were also obtained in lymphoma cells (data not shown). As demonstrated by both flow cytometry and confocal microscopy, 27 μM of LTX-401 induced a decreased signal in B16F1 melanoma cells after 60 minutes of incubation.

### 3 Discussion

The ability of cancer cells to evade immunosurveillance has recently been acknowledged as an emerging hallmark of cancer. Immunogenic cell death (ICD) is defined as the release of immune-potentiating molecules termed danger-associated molecular pattern molecules (DAMPs), and thus is critical in establishing an immune response against cancer cells. Due to the ability of some cytostatic compounds to induce ICD, it has also been shown that the immune system plays an important role in cancer eradication as a response to conventional treatment. ICD has additionally been suggested as a determinant for the long-term success of anticancer therapies. Oncolytic therapy is a new and promising therapeutic approach against solid tumors, where cancer cells are lysed in situ with the subsequent release of DAMPs.

The recently designed amino acid derivative LTX-401 has been reported to exhibit anticancer activity. In the present study, we demonstrate that LTX-401 exerts anticancer activity against a range of cancer cell lines, including B16 melanoma. We have previously designed shorter peptides with the potential to adopt an a-helical coil structure based on structure-activity relationship studies (SAR) on bovine lactoferricin (LfcinB) derivatives. These peptides have been shown to kill cancer cells more effectively than the naturally occurring LfcinB (25-mer), both in vitro and in vivo. SAR studies revealed that the size of the aromatic sector, and hence the overall higher hydrophobicity, is an important factor that will potentiate the anticancer activity of the peptide (FIG. 11).

Kinetic experiments were conducted in order to study the impact of different concentrations of LTX-401 against B16F1 melanoma cells over time. The concentrations used in the kinetic studies represented the 2×IC₅₀^{4h} and 4×IC₅₀^{4h} values. The cellular survival was reduced to a minimum after 90-120 min of using the 4×IC₅₀^{4h} value (108 μM) (FIG. 12). By contrast, commonly used chemotherapeutic agents require longer incubation periods to exert a significant anticancer effect.

To study the morphological changes of LTX-401 treated cancer cells, transmission electron microscopy (TEM) studies were performed. These studies revealed an early loss of surface morphology and a slight vacuolization of the cytoplasm of treated B16F1 cells (FIG. 13c, d). An increase in cell size was also evident shortly after the start of LTX-401 treatment. By prolonging the incubation period of treated cells (to 60 min), TEM images demonstrated an increased vacuolization, and revealed that cells were killed by a lytic mode of action that resulted in a loss of cell membrane integrity and the subsequent leakage of intracellular contents into the extracellular milieu (FIG. 13e, f).

The formation of vacuoles containing cytoplasmic material may constitute a transitional state in which cells respond to acute intracellular stress exerted by LTX-401 on different organelles The mitochondria displayed normal morphology five minutes post treatment, while showing evidence of swelling at experimental endpoint (60 min). Cell lines derived from the B16 cell line are known to consist of a heterogeneous population of both spindle-shaped and epithelial-like cells. These different phenotypes could possibly respond differently to treatment with anticancer substances, including LTX-401, which may partially explain the heterogeneous morphology of the treated cells.

When the concept of immunogenic cell death (ICD) was introduced, it was recognized that the mode of cancer cell death plays an important role in determining the outcome and success of selected anticancer therapies, including radiotherapy and several commonly used chemotherapeutic regimens. The activation of potent anti-tumor immune responses has been shown to rely on a series of cellular and biochemical events culminating in the release of DAMPs from dying and/or stressed tumor cells, including surface-exposed calreticulin (CRT), secreted adenosine triphosphate (ATP) and passively released High Mobility Group Box-1 protein (HMGB1). These three molecules are considered the hallmarks of ICD. When interacting with their respective receptors, DAMPs, along with tumor antigens, may orchestrate the recruitment and activation of dendritic cells (DCs) into the tumor bed, which may later home to draining lymph nodes to active tumor-specific CD8⁺ T cells. In the present study, B16F1 melanoma cells treated with LTX-401 in vitro were screened for the release of ATP and HMGB1 using a luciferase assay and Western blot analysis, respectively. These experiments revealed that LTX-401 treatment induced the release of HMGB1 and ATP in B16F1 cells (FIGS. 14a, c). The translocation of HMGB1 from the intracellular compartment into the supernatant was evident in B16F1 cells treated with LTX-401. The extracellular release of HMGB1 from post-apoptotic and/or necrotic cells is capable of sustaining and augmenting an anti-tumorigenic environment by the attraction of inflammatory leukocytes and the stimulation of pro-inflammatory cytokines such as TNF-α and IL-6. In addition, HMGB1 serves a pivotal role in the maturation of DCs and favors both processing and the presentation of tumor antigens to naïve T cells, thereby establishing a link between innate and adaptive anti-tumor immune responses.

When an ATP is released or secreted into the extracellular milieu by tumor cells, it acts on purinergic receptors to help facilitate the recruitment of immune cells into the tumor bed. Moreover, when binding to P₂X₇ receptors on DCs, ATP stimulates the assembly of NLRP3 inflammasome, and initiates a series of downstream events that ultimately result in the production and release of IL-1β, a cytokine required for the priming of IFN-γ producing tumor-specific CD8⁺ T cells. ATP was released from B16F1 in an increasing manner during the experimental time period. Mitochondrial DAMPs (mtDAMPs) include ATP, mitochondrial DNA, formyl peptides, oxidized cardiolipin and cytochrome c. These molecules are considered to be potent immune activators, as mitochondria bears a striking resemblance to bacteria. Cytochrome c marks one of the early events during apoptotic cell death, in which its release from the mitochondrial intermembrane space into the cytosol controls the assembly of the apoptosome and activation of procaspase-9, thus acting like an intracellular danger signal. The release of cytochrome c is also reported to occur from cells succumbing to necrosis. Furthermore, extracellular cytochrome c induces the activation of NF-kB and the release of other proinflammatory cytokines and chemokines. Elevated serum levels of cytochrome c have been observed in SIRS patients and are linked to poor survival. A cytochrome c ELISA assay was performed to help assess the capability of LTX-401 to release mtDAMPs. Treatment with LTX-401 was shown to induce the extracellular release of cytochrome c from B16F1 melanoma cells in vitro (see FIG. 14b). Interestingly, while TEM micrographs confirmed the lysis of B16F1 cells after 60 min, the release of cytochrome c was not significantly different from controls until after 120 min, which could indicate that the mitochondria is still somewhat intact and continues to retain its cytochrome c for a while after cell lysis.

The electron microscopy studies support the notion that mitochondria in B16F1 cells are not initially affected by LTX-401 treatment, and the timing of cytochrome c release could imply that the lysis of the mitochondria is secondary to the lysis of the cell. Previous mode of action studies by Ausbaucher et al. supports these findings, in which LTX-401 did not compromise the mitochondrial membrane potential as measured by TMRE. However, LTX-401 is a small molecule with an amphipathic structure, thus possessing the potential to bypass the plasma membrane, subsequently targeting intracellular structures. We therefore wanted to investigate other potential intracellular targets involved. The effect of LTX-401 treatment on acidic organelles was assessed by the use of lysosomal dye LysoTracker DND-26, with the confocal imaging of live cells demonstrating that LTX-401 treatment significantly altered the fluorescence of the acidophilic dye LysoTracker, even before gross morphological changes occurred. This observation was confirmed using flow cytometry analysis with the same concentrations and incubation time (data not shown). The loss of fluorescence indicates that acidic organelles in the cells are compromised due to treatment. Even so, B16F1 cells also harbor melanosomes, which are acidic lysosome-related organelles. Consequently, we repeated the experiment in a non-melanocytic cell line (A20, murine lymphoma), and achieved similar results (data not shown). These findings suggest that the lysosomes are among the intracellular targets of LTX-401.

Immunotherapeutic strategies aim to mount a specific T-cell response against tumor cells. Intratumoural immunotherapy for melanoma is a promising approach, with several preclinical and clinical trials reporting exciting results. In our study, we have investigated the anticancer efficacy and mode of action of a small lytic amino-acid derivative LTX-401. Melanoma cells treated with LTX-401 demonstrated features of immunogenic cell death, as shown by the release of DAMPs such as ATP, HMGB1 and cytochrome c. Furthermore, LTX-401 induced complete regression of highly aggressive and poorly immunogenic murine B16 melanomas. In conclusion, our results demonstrate the potential of LTX-401 as a promising immunotherapeutic agent.

### Example 4

### LTX-315 Increases T Cell Clonality in Murine Melanoma Model

### 1 Materials and Methods

This study was carried out using an “immunoSEQ™” T cell receptor (TCR) repertoire characterisation platform by Adaptive Biotechnologies™. The platform combines novel multiplex PCR1 with highly optimized primer sets and deep sequencing techniques that exclusively target TCR genes. The immunoSEQ platform enables researchers to analyse the adaptive immune system with exceptional depth and specificity.

Most TCR sequences will be found in only one or a few cells in an individual. During the formation of immunological memory, however, cells undergo clonal expansion. Consequently, after expansion and conversion to memory cells, receptor sequences against past pathogens can be present in thousands of cells. The large potential diversity of receptor sequences means that in most cases, nucleotide-identical sequences are not shared between cells except through clonal expansion.

By quantitating the exact number of input cells that contribute to an observed sequence, the immunoSEQ Assay allows researchers to analyse features of the highly expanded clones and also of infrequent (unique) cells.

### 1.1 Tumour Treatment

Murine B16 melanoma cells were harvested, washed and injected intradermally in ten mice. 8 days after tumour injection, peptide treatment was initiated using single intratumoural injections of LTX-315 (1.0 mg LTX-315/50 μl saline) once or twice in five mice. Vehicle control of saline only (0.9% NaCl in sterile H₂O) was administered to the other five mice. Animals were then euthanized on day 15.

### 1.2 TCR Repertoire Characterisation

Tumour tissue (10 mg) and whole blood (150-200 μl) was taken from each mouse for TCR repertoire characterisation analysis. Blood samples provide information regarding the immune repertoire in the periphery, whilst the tumour samples provide a focused view of the repertoire.

Sequence analysis was carried out by Adaptive Biotechnologies™ at a survey-level resolution with respect to the tissue samples and at a deep-level resolution with respect to the blood samples.

### 1.3 Data Analysis

Clonality quantitates the extent of mono-or oligoclonal expansion by measuring the shape of the clone frequency distribution. Values range from 0 to 1, where values approaching 1 indicate a nearly monoclonal population. FIG. 15 provides example distribution curves of populations with a clonality of 0.05 (A) and of 0.32 (B).

Clonality is calculated using the following formulae:

$$\begin{matrix}Diversity = H = - {\sum }_{i = 1}^{N} {p}_{i} {log}_{2} ( {p}_{i} ) \\ Clonality = 1 - \frac{H}{{log}_{2} ( N )}\end{matrix}$$

p values were calculated using a Mann-Whitney U test.

### 2 Results

A summary of the results is shown in Table 2 below.

TABLE 2

| | | | | TCRs (T cell | |
|---|---|---|---|---|---|
| | TCR | | | estimate) | |
| | Template | Unique | | per nucleated | Input |
| | Molecules | TCRs | Clonality | cell (%) | DNA |
| Blood | — | — | — | — | |
| Treated | 135,283 | 116,120 | 0.023 | 17.4 | 5,316 |
| Untreated | 212,909 | 185,301 | 0.016 | 20.4 | 6,256 |
| Tissue | — | — | — | — | |
| Treated | 12,331 | 5,414 | 0.105 | 3.01 | 2,666 |
| Untreated | 982 | 665 | 0.033 | 0.24 | 2,666 |

A robust dataset was produced with more than 100,000 molecules detected in blood. A significant increase is clonality (FIG. 16A), number of T cells per nucleated cell (FIG. 16B) and T cell clone count (FIG. 17) was seen in tumours following treatment with LTX-315 compared to control (p<0.05). Although no significant increase in clonality was seen in blood following treatment compared to control (FIG. 18, p<0.05), FIGS. 19 and 20 show that, following treatment, T cell clones are more abundant in the periphery compared to tumour tissue. This observation is more prominent in FIG. 20.

### Example 5

### LTX-315 Induces Protective Immune Responses

Animals cured by LTX-315 treatment were protected against a re-challenge with live B16 tumour cells both intradermally and intravenously. Together, the data indicate that intratumoural treatment with LTX-315 can provide local tumour control followed by protective immune responses and has potential as a new immunotherapeutic agent.

### 1 Materials and Methods

The study was carried out as described in CAMILIO KA et al., Cancer Immunol. Immunother. 2014, 63: 601-13.

### 1.1 Reagents

LTX-315 and LTX-328 (K-A-Q-Dip-Q-K-Q-A-W-NH2) were purchased on request from Bachem AG (Bubendorf, Switzerland) and Innovagen (Lund, Sweden), respectively. Dacarbazine (D2390), temozolomide (T2577) and cisdiammineplatinum dichloride (P4394) were all purchased from Sigma-Aldrich.

### 1.2 Cell Lines

B16F1 (AT CC, CRL-6323), a skin malignant melanoma of C57BL/6 murine origin, MRC-5 (AT CC, CCL-171), a human embryonic lung fibroblast cell line and HUV-EC-C (AT CC, CRL-1730), a human umbilical vein endothelial cell line, were all purchased from the American Type Culture Collection (AT CC-LGC Standards, Rockville, MD, USA). A375 (ECACC, 88113005) is a human malignant melanoma derived from patient material purchased from Public Health England (PHE Culture Collections, Porton Down, Salisbury, UK). B16F1 and A375 cells were cultured in DMEM (high glucose) and MRC-5 cells in MEM (normal glucose) containing 2 mM I-glutamine (all) and 1% non-essential amino acids (A375 only). Primary epidermal melanocytes (AT CC, PCS-200-013) were cultured in Dermal Basal Medium (ATCC, PCS-200-030) supplemented with the Adult Melanocyte Growth Kit (ATCC, PCS-200-042). HUV-EC-C was cultured using the EGM-2 BulletKit from Lonza, and all growth media were without antibiotics and supplemented with 10% FBS (except serum-free primary melanocytes). Cell cultures were maintained in a humidified atmosphere of 5% CO₂ and >95% humidity at 37° C. and tested for either both mycoplasma and other pathogens (Rapid-MAPTM-27, Taconic, Europa) or mycoplasma alone.

### 1.3 Animals

Female C57BL/6 wild-type mice, 5-6 weeks old, were obtained from Charles River, United Kingdom. All mice were housed in cages in a pathogen-free animal facility according to local and European Ethical Committee guidelines.

### 1.4 Tumour Treatment

Tumour cells were harvested, washed in RPMI-1640 and injected intradermally (i.d.) into the right side of the abdomen in C57BL/6 mice (5×10⁴ B16F1 cells per mouse/50 μl RPMI-1640). Palpable tumours (20-30 mm²) were injected i.t. with single doses of LTX-315 or LTX-328 dissolved in saline (1.0 mg peptide/50 μl saline) once a day for 3 consecutive days, and the vehicle control was saline only (0.9% NaCl in sterile H2O). Tumour size was measured using an electronic caliper and expressed as the area of an ellipse [(maximum dimension/2)×(minimum dimension/2)×π]. Animals were then euthanized when the product of the perpendicular tumour dimensions reached 130 mm² or when tumour ulceration developed.

### 1.5 Secondary Tumour Challenge

Animals with a complete regression (CR) of tumour after LTX-315 treatment were given a second i.d. tumour cell challenge (5×10⁴ B16F1 cells) on the left-hand side of the abdomen (contralateral to the first tumour site) 4-5 weeks after they were cured by LTX-315. Animals surviving the i.d. tumour re-challenge were later given a second tumour rechallenge intravenously (i.v.) (2×10⁵ cells) through the tail vein. Lungs were harvested on day 19 after the i.v. re-challenge. All mice were monitored for tumour size and survival.

### 1.6 Statistical Analysis

All data represent at least three independent experiments and are expressed as the mean±the standard deviation (SD) or the standard error of mean (SEM). Animal survival curves (Kaplan-Meier Plot) were compared using a log-rank (Mantel-Cox) test. We considered p values ≤0.05 to indicate statistical significance.

### 2 Results

To examine whether treatment with LTX-315 was able to induce adaptive immune responses, cured animals (n=25), together with non-treated control animals (n=6), were re-challenged with 5×104 viable tumour cells i.d. on the abdomen contralateral to the first tumour site 4-5 weeks after CR was attained. Animals achieving CR following the i.d. tumour re-challenge were later re-challenged a second time i.v. The majority of animals cured by LTX-315 showed growth inhibition, and tumour growth was absent in 15 out of the 25 animals, while all the control animals developed tumours subsequent of i.d. re-challenge (FIG. 21c). Animals previously cured by LTX-315 i.t. injections (n=7) showed systemic immune protection against B16 melanomas following i.v. re-challenge (FIG. 21d, e), compared to non-treated control animals (n=7). LTX-315-cured animals (mean # tumour foci=15.86) displayed a significantly lower number of lung tumour foci compared to non-treated control animals (mean #tumour foci=123.3). Additionally, histological examinations revealed that the lung tumour foci of animals previously cured by LTX-315 were significantly more infiltrated by CD3+ T cells, compared to the less infiltrated lung tumour foci of non-treated control animals (FIG. 21f).

As presented in patent publication WO 2007/107748, a similar adaptive immunity response was observed after administration with the lytic peptides LfcinB (N₂N-FKCRRWQWRMKKLGAPSITCVRRAF-COOH), Model 28 (H₂N-KAAKKAAKAbipKKAAKbipKKAA-COOH), Model 39 (H₂N-WKKWdipKKWK-COOH) in D and L form and C12 (H₂N-KAAKKAbipKAAKAbipKKAA-COOH). Bip=biphenylalanine.

### 3 Discussion

To investigate whether the immune-modulating properties of LTX-315 in vivo led to a long-term protective immune response, animals were re-challenged with live tumour cells. All previously cured animals demonstrated significant tumour growth inhibition and no tumour take was observed in 60% of the animals given viable cells i.d. (FIG. 21a-c). Animals surviving the i.d. re-challenge were re-challenged with viable B16 melanoma cells i.v. 9 months post-LTX-315 treatment to examine a possible long-lasting systemic protection against the cancer and its effect in an experimental lung metastasis model. All animals re-challenged i.v. demonstrated systemic immune protection against B16 melanomas compared to non-treated control animals (FIG. 21d, e) in addition to an increase in the amount of infiltrating CD3+ T cells into the re-challenged lung tumour foci, as seen by immunolabelling with anti-CD3 (FIG. 21f). This indicates that LTX-315 induced systemic antitumor immune responses, with the persistence of a T cell-mediated antitumor immunity and thus a long-term protection against relapse of the tumour and against lung tumour foci. The mechanism of LTX-315 is thought to be by inducing long-term, specific cellular immunity against B16 melanomas through membrane-induced lysis and the extracellular release of DAMPs (HMGB1). Taken together, our observations in vitro and in vivo indicate that i.t. administration of LTX-315 leads to extensive tumour necrosis initiated by a direct disruptive effect of the peptide on the plasma membrane of tumour cells. Moreover, the necrotic effect of LTX-315 leads to the release of DAMPs that stimulates immune responses and the infiltration of TILs into the tumour parenchyma, which may be critical in the eradication of solid B16 melanomas due to their possible role in inducing a long-lasting tumour immune protection.

### Example 6

### T Cells Generated After LTX-315 Administration Play a Key Role in T Cell Regression

Here, we show that LTX-315 rapidly reprograms the tumour microenvironment by increasing the frequency of polyfunctional T helper type 1/type 1 cytotoxic T cells. This increase plays an important role in tumour regression.

### 1 Materials and Methods

### 1.1 Chemicals and Cell Cultures

Media and supplements for cell culture were obtained from Gibco-Life Technologies (Carlsbad, CA, USA), chemicals from Sigma-Aldrich (St. Louis, MO, USA) with the exception of LTX-315 that was provided by Lytix Biopharma (Tromsø, Norway) and plasticware from Corning BV Life Sciences (Amsterdam, The Netherlands). MCA205 was cultured in RPMI-1640 medium supplemented with 10% fetal calf serum, and 2 mM I-glutamine, 100 IU/ml penicillin G sodium salt, 100 μg/ml streptomycin sulfate, 1 mM sodium pyruvate and 1 mM non-essential amino acids. Cells were grown at 37° C. in a humidified incubator under a 5% CO₂ atmosphere.

### 1.2 Mice

Mice were maintained in specific pathogen-free conditions in a temperature-controlled environment with 12-h light, 12-h dark cycles and received food and water ad libitum. Animal experiments followed the Federation of European Laboratory Animal Science Association (FELASA) guidelines, were in compliance with EU Directive 63/2010 and were approved by the Ethical Committee of the Gustave Roussy Cancer Campus (Villejuif, France). Mice were used between 7 and 14 weeks of age. WT-specific pathogen-free (SPF) C57BL/6 J were obtained from Envigo (Gannat, France) and were kept in SPF conditions at Gustave Roussy, Villejuif, France.

### 1.3 Tumour Models

Mice were subcutaneously injected into the right flank with 1×10⁶ MCA205 cells. Tumour cell lines were inoculated into C57BL/6 mice. Tumour surfaces (longest dimension×perpendicular dimension) were routinely monitored by caliper. When tumours reached a size of 20-25 mm² (day 0), mice were administered intratumourally with three consecutive daily injections of 300 μg LTX-315. In T-cell depletion experiments (FIG. 24), anti-CD4 and anti-CD8 monoclonal antibodies (mAbs) (GK1.5 and 53-6.72, respectively; 200 μg per mouse) or their isotype controls (LTF-2 and 2A3, respectively) were injected intraperitoneally 3 and 4 days before the first LTX-315 injection and continued every other 7 days. All mAbs for in vivo use were obtained from BioXcell (West Lebanon, NH, USA), using the recommended isotype control mAbs.

### 1.4 Flow Cytometry

Tumours and spleens were harvested 7 days after the first injection of LTX-315. Excised tumours were cut into small pieces and digested in RPMI-1640 medium containing Liberase at 25 μg/ml (Roche, Boulogne-Billancourt, France) and DNase1 at 150 UI/ml (Roche) for 30 min at 37° C. The mixture was subsequently passaged through a 100 μm cell strainer. 2×10⁶ splenocytes (after red blood cells lysis) or tumour cells were preincubated with purified anti-mouse CD16/CD32 (93;eBioscience, San Diego, CA, USA) for 15 min at 4° C., before membrane staining. For intracellular staining, the FoxP3 staining kit (eBioscience) was used. Dead cells were excluded using the Live/Dead Fixable Yellow dead cell stain kit (Life Technologies, Carlsbad, CA, USA). For cytokine staining, cells were stimulated for 4 h at 37° C. with 50 ng/ml of phorbol 12-myristate 13-acetate (PMA; Calbiochem, San Diego, CA, USA), 1 μg/ml of ionomycin (Sigma, St. Louis, MO, USA), and BD Golgi STOP (BD Biosciences, San Jose, CA, USA). Anti-IFN-γ (XMG1.2) and anti-TNF-α (MP6-XT22) were purchased from eBioscience. Anti-CD4 (GK1.5) were anti-CD8β (YTS1567.7) were purchased from Biolegend (San Diego, CA, USA). Eight-colour flow cytometry analysis was performed with antibodies conjugated to fluorescein isothiocyanate, phycoerythrin, phycoerythrin cyanin 7, peridinin chlorophyll protein cyanin 5.5, allophycocyanin cyanin 7, Pacific blue or allophycocyanin. All cells were analysed on a CyAn ADP (Beckman Coulter, Marseille, France) flow cytometer with FlowJo (Tree Star, Ashland, OR) software.

### 1.5 Statistical Analysis

Data were analysed with Microsoft Excel (Microsoft Co., Redmont, WA, USA) and Prism 5 (GraphPad, San Diego, CA, USA). Data are presented as means±S.E.M. and P-values computed by unpaired Student's t-tests or one-way ANOVA followed by Tukey's test where applicable. Comparisons of Kaplan-Meier survival curves were performed using the log-rank Mantel-Cox test. All reported tests are two-tailed and were considered significant at P-values <0.05.

### 2 Results

We explored the dynamics of the main effectors composing the tumour microenvironment shaped 7 days post LTX-315 in a subcutaneous sarcoma model. We observed an accumulation of IFNγ⁺ (T helper type 1, Th1), IL-17⁺ (Th17) and double-positive IFNγ⁺ IL-17⁺ (pTh17) CD4⁺ TILs (FIG. 22), as well as polyfunctional IFNγ⁺ TNFα⁺ CD8⁺ T cells (FIG. 23).

LTX-315-mediated anticancer effects were T-cell dependent in as much as antibodies depleting CD4⁺ and CD8⁺ T cells completely abrogated the antitumor effects (FIG. 24).

### 3 Discussion

In this preclinical study, we found that LTX-315, whilst not inducing a typical apoptotic or a regulated necrotic cell death, causes uncontrolled, immunogenic tumour cell death. At least part of this cell death includes facilitating the accumulation of polyfunctional CD4⁺ and CD8⁺ TILs. The immunogenic mechanism of action is shown to be important by the fact that antitumor effects were abolished in the absence of T lymphocytes.

### Example 7

### Clinical Histology Evidence of T Cell Infiltration Into Tumour After LTX-315 Administration

### 1 Materials and Methods

Formalin-fixed and paraffin-embedded tumour tissue sections from patients were deparaffinized in xylene and graded alcohols, hydrated and washed in PBS. After antigen retrieval in sodium citrate buffer (pH 6) in a microwave oven, the endogenous peroxidase was blocked by 0.3% H₂O₂ for 15 min. Sections were incubated overnight at 4° C. with primary antibody; rabbit polyclonal anti-CD3 (clone A0452 Dako) or mouse monoclonal anti-CD8 (clone OX8, ab33786, Abcam). As a secondary antibody, the anti-rabbit-horseradish peroxidase (HRP) SuperPicTure Polymer detection kit (Invitrogen) or Envision system HRP-anti-mouse (Dako) were used. A matched isotype control was used as a control for nonspecific background staining

### 2 Results

FIGS. 25 to 29 show infiltration of cytotoxic T cells into the tumours of metastatic melanoma, malignant melanoma, myo-epithelioma, breast carcinoma and desmoid tumour patients.

### Example 8

### LTX-315 Treatment Leads to Cytotoxic T Cell Infiltration Into Secondary as Well as Primary Tumours

To clarify whether intratumoural injection of LTX-315 in one tumor lesion could also have an effect on metastatic disease, intraperitoneal tumour and two subcutaneous tumours were established in a rat sarcoma model. Thereafter, LTX-315 was injected into one of the subcutaneous lesion and tumor growth assessed by live imaging. The results showed that LTX-315 treatment eradicated all three lesions and the animals went into durable complete remission.

### 1 Materials and Methods

### 1.1 Animal Experiments

Rats of the inbred Piebald Virol Glaxo (PVG.RT7a, abbreviated PVG) strain were used interchangeably with the PVG.RT7b strain (in this study abbreviated PVG). The strains are identical except for one irrelevant epitope of the leukocyte common antigen (LCA/CD45) family. PVG rats were purchased from Harlan (the Netherlands) and PVG.7B rats were obtained from in-house breeding at the Institute of Basic Medical Sciences (IMB, University of Oslo, Norway). During the experiments, male rats, weighing 240-270 g, were kept in groups of 2 to 3 animals per cage under climate-controlled conditions, with 12 h light/dark cycles and ambient temperature. The rats were housed in an enriched individually ventilated cage (IVC) system with free access to standard rodent chow and water ad libitum. The animals were anesthetized during the experimental procedures with either 2.5% Isofluran gas (Baxter Medical AB) or received subcutaneous injections (0.4 ml/kg) of fentanyl/fluanisonone (Hypnorm; VetaPharma Ltd.), which provided sufficient degree of sedation and analgesia. The animals were monitored daily and large-tumor-bearing rats were euthanized with CO₂. All procedures performed were conducted under FOTS number 1957 and 5917 and approved by the Experimental Animal Board under the Ministry of Agriculture of Norway and in compliance with The European convention for the Protection of Vertebrate Animals used for

Experimental and other Scientific Purposes. The laboratory animal facilities are subject to a routine health monitoring program and were screened for common pathogens according to a modification of the Federation of European Laboratory Animal Science Association recommendation.

### 1.2 Tumour Treatment

Pre-cultured rat transformed mesenchymal stem cell-derived sarcoma model cells (rTMSCs) were harvested in serum free RPMI-1640 and 200,000 rTMSCs were subcutaneously inoculated in the right flank into PVG rats on day ÷2 and 20,000 rTMSCs in the opposite flank on day 0. Established tumours (25 mm² mean tumour size) were injected intralesionally with LTX-315 (dissolved in sterile H₂O with 0.9% NaC ) (n=8) or with vehicle (sterile H₂O with 0.9% NaCl) (n=6). Treatment doses of LTX-315, 50 μl at 20 mg/ml (1 mg), were given using a 1 ml syringe (Myjector U-100, Terumo) needle 0.5×16 mm (Fine-Ject; Henke-Sass, Wolf GmbH. Injections were provided daily for three subsequent days. Tumour size was measured three times a week using a caliper and expressed as the area of an ellipse [(maximum dimension/2)×(minimum dimension/2)]. Animals were terminated when the tumor exceeded 400 mm².

### 1.3 Preparation of Single Cell Suspensions From Solid Tumours

Single-cell suspensions were prepared from fresh tumour tissue at different time points during the week after LTX-315 treatment. Tumours were gently minced with a razor blade and cut into small pieces (4 mm²). Tumour tissue was incubated with Liberase TM (Thermolysin Medium; Roche Diagnostics) at a concentration of 0.18 Wünsch units/ml in 10 ml MEM media (Sigma-Aldrich) at 37° C. for 60 min with gentle agitation. The enzymatic digestion was terminated by addition of 2 ml 4° C. FCS (Invitrogen, Thermo Fischer). The cell suspension was filtered through a 70 μM mesh (Cell Strainer; BD), washed in PBS and cells were then directly used for flow cytometric staining.

### 1.4 Antibodies and FACS Analysis

The mouse monoclonal antibodies against CD4 (OX38) and CD8 (OX38) were isolated from culture supernatants from hybridomas and were kind gifts from the MRC Cellular Immunology Unit, Oxford, UK. They were conjugated at IMB according to standard protocols. Fluorochrome-conjugated mAbs against CD3 (G4.18) was obtained from BD Biosciences, including PerCP Streptavidin. A four-color panel consisting of reagents in FITC/AI488, PE, PerCP Streptavidin and AI647 was applied and analyzed on a FACS Calibur (BD) equipped with the CellQuest software (BD). Dot plot and histogram gates were set using isotype control antibodies.

### 1.5 Immunohistochemistry

Formalin-fixed and paraffin-embedded tissue sections were deparaffinized in xylene and graded alcohols, hydrated and washed in PBS. After antigen retrieval in sodium citrate buffer (pH 6) in a microwave oven, the endogenous peroxidase was blocked by 0.3% H₂O₂ for 15 min. Sections were incubated overnight at 4° C. with primary antibody; rabbit polyclonal anti-CD3 (clone A0452 Dako) or mouse monoclonal anti-CD8 (clone OX8, ab33786, Abcam). As a secondary antibody, the anti-rabbit-horseradish peroxidase (HRP) SuperPicTure Polymer detection kit (Invitrogen) or Envision system HRP-anti-mouse (Dako) were used. A matched isotype control was used as a control for nonspecific background staining.

### 1.6 Statistics

Data are expressed as mean±SD. Statistical differences between two groups were analyzed by two-tailed Student's t test, P<0.05 was considered to be statistically significant. Statistical analyses were performed using GraphPad Prism software (version 6, GraphPad).

### 2 Results

In order to investigate the cellular mechanisms underlying LTX-315-mediated regression and long term protective immune responses, we analysed the cellular composition of treated tumours by flow cytometry and immunohistochemistry. During the week after the last treatment, tumours were resected at different time points and thereafter tumor infiltrating leukocytes were phenotyped in both LTX-315-treated and untreated animals (FIG. 30a).

Tumor infiltrating T lymphocytes in response to growing tumours were observed in untreated rats (FIG. 30b). The spontaneous infiltration of lymphocytes was insufficient to inhibit tumour growth and tumour control was dependent of adaptive immunity and accumulation of CD8⁺ T cells in the tumour microenvironment. The percentage of T cells was significantly increased after LTX-315 treatment compared to untreated tumors (treated; 28.35±11.78, untreated; 13.58±7.84, P<0.5). Similarly, the percentage of T cells within secondary tumor tissue increased 3-fold compared to untreated rats (treated; 39.83±17.4, untreated; 11.83±10.25, P<0.01). Within the CD3⁺ T cell population, CD8⁺ cells accounted for the major fraction in primary and secondary tumors of treated rats versus untreated controls (primary tumor of treated rats; 62.77±13.3, untreated rats; 37.33±3.48, P<0.01, secondary tumor of treated rats; 45.8±5.4, untreated rats; 34.67±3.36, P<0.1).

Compared to untreated rats, FACS analysis of LTX-315 treated rats revealed significantly elevated levels of CD3⁺ and CD8⁺ tumor infiltrating T cells, the major immune effector cell population, that correlated with tumor regression.

Immunohistochemical analysis was consistent with the flow cytometry data demonstrating an increased infiltration of CD3⁺ and CD8⁺ cells observed in primary and secondary tumor tissues from LTX-315 treated rats (FIG. 31).

1-17. (canceled)

18. A method of treating tumour cells or preventing or reducing the growth, establishment, spread, or metastasis of a tumour, which method comprises administering a therapeutically effective amount of an unfractionated population of tumour-infiltrating T cells to a subject in need thereof, wherein the unfractionated population of T cells is not enriched for certain clonotypes, and wherein said unfractionated population is obtained by a method comprising:
isolating a population of T cells from a cellular tumour sample taken from said subject, wherein the subject has been treated with a positively charged amphipathic amino acid derivative or peptide which is able to lyse tumour cell membranes;
and wherein the peptide;
a) consists of 9 amino acids in a linear arrangement;
b) of those 9 amino acids, 5 are cationic and 4 have a lipophilic R group; and
c) at least one of said 9 amino acids is a non-genetically coded amino acid or a modified derivative of a genetically coded amino acid;
d) the lipophilic and cationic residues are arranged such that there are no more than two of either type of residue adjacent to one another; and
e) the peptide comprises two pairs of adjacent cationic amino acids and one or two pairs of adjacent lipophilic residues; or
wherein the amino acid derivative or peptide is 1 to 4 amino acids in length, has a net positive charge of at least +2 and incorporates a disubstituted β amino acid, each of the substituting groups in the β amino acid, which may be the same or different, comprises at least 7 non-hydrogen atoms, is lipophilic and has at least one cyclic group, one or more cyclic groups within a substituting group may be linked or fused to one or more cyclic groups within the other substituting group and where cyclic groups are fused in this way the combined total number of non-hydrogen atoms for the two substituting groups is at least 12.

19. The method of claim 18, wherein the method further comprises culturing said unfractionated T cell population to maintain or expand said population.

20. The method as claimed in claim 18, wherein the peptide contains at least two cyclic groups.

21. The method as claimed in claim 18, wherein the peptide comprises at least one lipophilic group of ten or more non-hydrogen atoms.

22. The method as claimed in claim 18, wherein the peptide is LTX-315 (SEQ ID NO: 23), or a salt thereof.

23. The method as claimed in claim 18, wherein the amino acid derivative is LTX-401 having the structure: 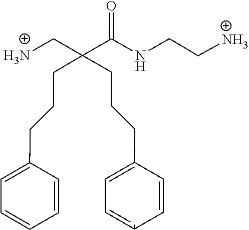
or a salt thereof.

24. The method as claimed in claim 18, wherein the unfractionated population of tumour-infiltrating T cells is administered with a checkpoint inhibitor.


SEQUENCE LISTING (48 sequences)

SEQ ID NO: 1 - protein, 9 aa, synthetic construct
  VARIANT 1..2: note = or R or H or any non-genetically coded or modified amino acid carrying a positive charge at pH 7.0
  VARIANT 5..6: note = or R or H or any non-genetically coded or modified amino acid carrying a positive charge at pH 7.0
  VARIANT 9: note = or R or H or any non-genetically coded or modified amino acid carrying a positive charge at pH 7.0
  VARIANT 3..4: note = W or a non-genetically coded lipophilic amino acid
  VARIANT 7..8: note = W or a non-genetically coded lipophilic amino acid
  VARIANT 1..9: note = three X positions are W and one is a non-genetically coded lipophilic amino acid
KKXXKKXXK

SEQ ID NO: 2 - protein, 9 aa, synthetic construct
  VARIANT 2..3: note = or R or H or any non-genetically coded or modified amino acid carrying a positive charge at pH 7.0
  VARIANT 6..7: note = or R or H or any non-genetically coded or modified amino acid carrying a positive charge at pH 7.0
  VARIANT 9: note = or R or H or any non-genetically coded or modified amino acid carrying a positive charge at pH 7.0
  VARIANT 1: note = W or a non-genetically coded lipophilic amino acid
  VARIANT 4..5: note = W or a non-genetically coded lipophilic amino acid
  VARIANT 8: note = W or a non-genetically coded lipophilic amino acid
  VARIANT 1..9: note = three X positions are W and one is a non-genetically coded lipophilic amino acid
XKKXXKKXK

SEQ ID NO: 3 - protein, 9 aa, synthetic construct
  VARIANT 1: note = or R or H or any non-genetically coded or modified amino acid carrying a positive charge at pH 7.0
  VARIANT 4..5: note = or R or H or any non-genetically coded or modified amino acid carrying a positive charge at pH 7.0
  VARIANT 8..9: note = or R or H or any non-genetically coded or modified amino acid carrying a positive charge at pH 7.0
  VARIANT 2..3: note = W or a non-genetically coded lipophilic amino acid
  VARIANT 6..7: note = W or a non-genetically coded lipophilic amino acid
  VARIANT 1..9: note = three X positions are W and one is a non-genetically coded lipophilic amino acid
KXXKKXXKK

SEQ ID NO: 4 - protein, 9 aa, synthetic construct
  VARIANT 1..2: note = or R or H or any non-genetically coded or modified amino acid carrying a positive charge at pH 7.0
  VARIANT 5: note = or R or H or any non-genetically coded or modified amino acid carrying a positive charge at pH 7.0
  VARIANT 8..9: note = or R or H or any non-genetically coded or modified amino acid carrying a positive charge at pH 7.0
  VARIANT 3..4: note = W or a non-genetically coded lipophilic amino acid
  VARIANT 6..7: note = W or a non-genetically coded lipophilic amino acid
  VARIANT 1..9: note = three X positions are W and one is a non-genetically coded lipophilic amino acid
KKXXKXXKK

SEQ ID NO: 5 - protein, 9 aa, synthetic construct
  VARIANT 1: note = or R or H or any non-genetically coded or modified amino acid carrying a positive charge at pH 7.0
  VARIANT 3..4: note = or R or H or any non-genetically coded or modified amino acid carrying a positive charge at pH 7.0
  VARIANT 7..8: note = or R or H or any non-genetically coded or modified amino acid carrying a positive charge at pH 7.0
  VARIANT 2: note = W or a non-genetically coded lipophilic amino acid
  VARIANT 5..6: note = W or a non-genetically coded lipophilic amino acid
  VARIANT 9: note = W or a non-genetically coded lipophilic amino acid
  VARIANT 1..9: note = three X positions are W and one is a non-genetically coded lipophilic amino acid
KXKKXXKKX

SEQ ID NO: 6 - protein, 9 aa, synthetic construct
  VARIANT 1..2: note = or R or H or any non-genetically coded or modified amino acid carrying a positive charge at pH 7.0
  VARIANT 5..6: note = or R or H or any non-genetically coded or modified amino acid carrying a positive charge at pH 7.0
  VARIANT 9: note = or R or H or any non-genetically coded or modified amino acid carrying a positive charge at pH 7.0
  SITE 3: note = a non-genetically coded lipophilic amino acid
KKXWKKWWK

SEQ ID NO: 7 - protein, 9 aa, synthetic construct
  VARIANT 1..2: note = or R or H or any non-genetically coded or modified amino acid carrying a positive charge at pH 7.0
  VARIANT 5..6: note = or R or H or any non-genetically coded or modified amino acid carrying a positive charge at pH 7.0
  VARIANT 9: note = or R or H or any non-genetically coded or modified amino acid carrying a positive charge at pH 7.0
  SITE 8: note = a non-genetically coded lipophilic amino acid
KKWWKKWXK

SEQ ID NO: 8 - protein, 9 aa, synthetic construct
  VARIANT 1..2: note = or R or H or any non-genetically coded or modified amino acid carrying a positive charge at pH 7.0
  VARIANT 5..6: note = or R or H or any non-genetically coded or modified amino acid carrying a positive charge at pH 7.0
  VARIANT 9: note = or R or H or any non-genetically coded or modified amino acid carrying a positive charge at pH 7.0
  SITE 4: note = a non-genetically coded lipophilic amino acid
KKWXKKWWK

SEQ ID NO: 9 - protein, 9 aa, synthetic construct
  VARIANT 2..3: note = or R or H or any non-genetically coded or modified amino acid carrying a positive charge at pH 7.0
  VARIANT 6..7: note = or R or H or any non-genetically coded or modified amino acid carrying a positive charge at pH 7.0
  VARIANT 9: note = or R or H or any non-genetically coded or modified amino acid carrying a positive charge at pH 7.0
  SITE 5: note = a non-genetically coded lipophilic amino acid
WKKWXKKWK

SEQ ID NO: 10 - protein, 9 aa, synthetic construct
  SITE 1: note = diphenylalanine
  MOD_RES 9: note = AMIDATION
XKKWWKKWK

SEQ ID NO: 11 - protein, 9 aa, synthetic construct
  SITE 5: note = diphenylalanine
  MOD_RES 9: note = AMIDATION
WKKWXKKWK

SEQ ID NO: 12 - protein, 9 aa, synthetic construct
  SITE 8: note = diphenylalanine
  MOD_RES 9: note = AMIDATION
WKKWWKKXK

SEQ ID NO: 13 - protein, 9 aa, synthetic construct
  SITE 1: note = biphenylalanine
  MOD_RES 9: note = AMIDATION
XKKWWKKWK

SEQ ID NO: 14 - protein, 9 aa, synthetic construct
  SITE 4: note = biphenylalanine
  MOD_RES 9: note = AMIDATION
WKKXWKKWK

SEQ ID NO: 15 - protein, 9 aa, synthetic construct
  SITE 5: note = D-diphenylalanine
  SITE 1: note = D-tryptophan
  SITE 2..3: note = D-lysine
  SITE 4: note = D-tryptophan
  SITE 6..7: note = D-lysine
  SITE 8: note = D-tryptophan
  SITE 9: note = D-lysine
  MOD_RES 9: note = AMIDATION
WKKWXKKWK

SEQ ID NO: 16 - protein, 9 aa, synthetic construct
  SITE 4: note = diphenylalanine
  MOD_RES 9: note = AMIDATION
KKWXKKWWK

SEQ ID NO: 17 - protein, 9 aa, synthetic construct
  SITE 4: note = diphenylalanine
  SITE 1..2: note = D-lysine
  SITE 5..6: note = D-lysine
  SITE 9: note = D-lysine
  MOD_RES 9: note = AMIDATION
KKWXKKWWK

SEQ ID NO: 18 - protein, 9 aa, synthetic construct
  SITE 4: note = diphenylalanine
  MOD_RES 9: note = AMIDATION
KKWXKKWXK

SEQ ID NO: 19 - protein, 9 aa, synthetic construct
  SITE 4: note = biphenylalanine
  MOD_RES 9: note = AMIDATION
KKWXKKWWK

SEQ ID NO: 20 - protein, 9 aa, synthetic construct
  SITE 2: note = biphenylalanine
  MOD_RES 9: note = AMIDATION
KXKKWWKKW

SEQ ID NO: 21 - protein, 9 aa, synthetic construct
  SITE 3: note = biphenylalanine
  MOD_RES 9: note = AMIDATION
KKXWKKWWK

SEQ ID NO: 22 - protein, 9 aa, synthetic construct
  SITE 7: note = diphenylalanine
  MOD_RES 9: note = AMIDATION
KKWWKKXWK

SEQ ID NO: 23 - protein, 9 aa, synthetic construct
  SITE 8: note = diphenylalanine
  MOD_RES 9: note = AMIDATION
KKWWKKWXK

SEQ ID NO: 24 - protein, 9 aa, synthetic construct
  SITE 3: note = diphenylalanine
  MOD_RES 9: note = AMIDATION
KWXKKWWKK

SEQ ID NO: 25 - protein, 9 aa, synthetic construct
  SITE 7: note = diphenylalanine
  MOD_RES 9: note = AMIDATION
KKWWKWXKK

SEQ ID NO: 26-27 - skipped (2 entries)

SEQ ID NO: 28 - protein, 9 aa, synthetic construct
  SITE 4: note = diphenylalanine
  MOD_RES 9: note = AMIDATION
RRWXRRWWR

SEQ ID NO: 29 - protein, 9 aa, synthetic construct
  SITE 6: note = diphenylalanine
  MOD_RES 9: note = AMIDATION
KWWKKXWKK

SEQ ID NO: 30 - protein, 9 aa, synthetic construct
  SITE 2: note = diphenylalanine
  MOD_RES 9: note = AMIDATION
KXKKWWKKW

SEQ ID NO: 31 - protein, 9 aa, synthetic construct
  SITE 3: note = diphenylalanine
  MOD_RES 9: note = AMIDATION
KKXWKKWWK

SEQ ID NO: 32 - protein, 9 aa, synthetic construct
  SITE 6: note = D-diphenylalanine
  SITE 1: note = D-lysine
  SITE 2..3: note = D-tryptophan
  SITE 4..5: note = D-lysine
  SITE 7: note = D-tryptophan
  SITE 8..9: note = D-lysine
  MOD_RES 9: note = AMIDATION
KWWKKXWKK

SEQ ID NO: 33 - protein, 9 aa, synthetic construct
  SITE 3: note = biphenylalanine
  MOD_RES 9: note = AMIDATION
RRXWRRWWR

SEQ ID NO: 34 - protein, 9 aa, synthetic construct
  SITE 3: note = diphenylalanine
  MOD_RES 9: note = AMIDATION
RRXWRRWWR

SEQ ID NO: 35 - protein, 9 aa, synthetic construct
  SITE 3: note = D-biphenylalanine
  SITE 1..2: note = D-lysine
  SITE 4: note = D-tryptophan
  SITE 5..6: note = D-lysine
  SITE 7..8: note = D-tryptophan
  SITE 9: note = D-lysine
  MOD_RES 9: note = AMIDATION
KKXWKKWWK

SEQ ID NO: 36 - protein, 9 aa, synthetic construct
  SITE 3: note = biphenylalanine
  SITE 1..2: note = D-lysine
  SITE 4: note = D-tryptophan
  SITE 5..6: note = D-lysine
  SITE 7..8: note = D-tryptophan
  SITE 9: note = D-lysine
  MOD_RES 9: note = AMIDATION
KKXWKKWWK

SEQ ID NO: 37 - protein, 9 aa, synthetic construct
  SITE 3: note = D-biphenylalanine
  MOD_RES 9: note = AMIDATION
KKXWKKWWK

SEQ ID NO: 38 - skipped

SEQ ID NO: 39 - protein, 9 aa, synthetic construct
  SITE 4: note = 1-naphthylalanine
  MOD_RES 9: note = AMIDATION
KKWXKKWWK

SEQ ID NO: 40 - protein, 9 aa, synthetic construct
  SITE 4: note = 2-naphthylalanine
  MOD_RES 9: note = AMIDATION
KKWXKKWWK

SEQ ID NO: 41 - protein, 9 aa, synthetic construct
  SITE 4: note = 2-amino-3-(anthracen-9-yl)propanoic acid
  MOD_RES 9: note = AMIDATION
KKWXKKWWK

SEQ ID NO: 42 - protein, 9 aa, synthetic construct
  SITE 4: note = 2-amino-3-[1,1':4',1"-terphenyl-4-yl]propionic acid
  MOD_RES 9: note = AMIDATION
KKWXKKWWK

SEQ ID NO: 43 - protein, 9 aa, synthetic construct
  SITE 4: note = diphenylalanine
  MOD_RES 9: note = AMIDATION
KAQXQKQAW

SEQ ID NO: 44 - protein, 25 aa, synthetic construct
FKCRRWQWRMKKLGAPSITCVRRAF

SEQ ID NO: 45 - protein, 20 aa, synthetic construct
  SITE 10: note = biphenylalanine
  SITE 16: note = biphenylalanine
KAAKKAAKAXKKAAKXKKAA

SEQ ID NO: 46 - protein, 9 aa, synthetic construct
  SITE 5: note = diphenylalanine
WKKWXKKWK

SEQ ID NO: 47 - protein, 9 aa, synthetic construct
  SITE 5: note = diphenylalanine
  SITE 1: note = D-tryptophan
  SITE 2..3: note = D-lysine
  SITE 4: note = D-tryptophan
  SITE 6..7: note = D-lysine
  SITE 8: note = D-tryptophan
  SITE 9: note = D-lysine
WKKWXKKWK

SEQ ID NO: 48 - protein, 17 aa, synthetic construct
  SITE 7: note = biphenylalanine
  SITE 13: note = biphenylalanine
KAAKKAXKAAKAXKKAA